(19) United States
(12) Patent Application Publication
(10) Pub. No.: US 2007/0185060 A1
(43) Pub. Date: Aug. 9, 2007

(54) Boronic acid thrombin inhibitors

(75) Inventor: Shouming Wang, London (GB)

Correspondence Address: KLARQUIST SPARKMAN, LLP, 121 SW SALMON STREET, SUITE 1600, PORTLAND, OR, 97204, US

(73) Assignee: TRIGEN LIMITED, LONDON (GB)

(21) Appl. No.: 10/591,868

(22) PCT Filed: Mar. 9, 2005
(86) PCT No.: PCT/GB05/00918

§ 371 Date: Sep. 6, 2006

(30) Foreign Application Priority Data
Mar. 9, 2004 (GB) 0405267.6

(51) Int. Cl. A61K 31/69; C07F 5/02

(52) U.S. Cl. 514/64; 548/405

(57) ABSTRACT

A thrombin inhibitor selected from boronic acids of formula (I), and salts, prodrugs and prodrug salts thereof: wherein X is H (to form NH₂) or an amino-protecting group; aa¹ is an amino acid residue having a side chain selected from formula (A) and (B)—(CO)ₐ—(CH₂)_{b}-D_{c}-(CH₂)_{d}-E (A), —(CO)ₐ—(CH₂)_{b}-D_{c}-Cₑ(E¹)(E²)(E³) wherein E¹, E² and E³ are 5-6 membered saturated or unsaturated hydrocarbyl rings, or one of E¹, E² and E³ is hydrogen and the other two are a said hydrocarbyl ring, E, E¹, E² and E³ optionally being halogenated when saturated and mandatorily being halogenated when unsaturated, a particular halogen being fluorine; aa² is a residue of an amino acid which binds to the thrombin S2 subsite; and R⁹ is a straight chain alkyl group interrupted by one or more ether linkages or R⁹ is —(CH2)mW and W is —OH or halogen.

### BACKGROUND

The present disclosure relates to pharmaceutically useful products obtainable from organoboronic acids. The disclosure also relates to the use of members of the aforesaid class of products, to their formulation, their preparation, their synthetic intermediates and to other subject matter.

The disclosure further relates to pharmaceutical formulations containing the described products.

Boropeptide Serine Protease Inhibitors

Shenvi (EP-A-145441 and U.S. Pat. No. 4,499,082) disclosed that peptides containing an α-aminoboronic acid with a neutral side chain were effective inhibitors of elastase and has been followed by numerous patent publications relating to boropeptide inhibitors of serine proteases.

In describing inhibitors or substrates of proteases, P1, P2, P3, etc. designate substrate or inhibitor residues which are amino-terminal to the scissile peptide bond, and S1, S2, S3, etc., designate the corresponding subsites of the cognate protease in accordance with: Schechter, I. and Berger, A. On the Size of the Active Site in Proteases, Biochem. Biophys. Res. Comm., 27:157-162, 1967. In thrombin, the S1 binding site or “specificity pocket” is a well defined slit in the enzyme, whilst the S2 and S3 binding subsites (also respectively called the proximal and distal hydrophobic pockets) are hydrophobic and interact strongly with, respectively, Pro and (R)-Phe, amongst others.

Aminoboronate or peptidoboronate inhibitors or substrates of serine proteases are described in:
U.S. Pat. No. 4,935,493
EP 341661
WO 94/25049
WO 95/09859
WO 96/12499
WO 96/20689
Lee S-L et al, Biochemistry 36:13180-13186, 1997
Dominguez C et al, Bioorg. Med. Chem. Lett. 7:79-84, 1997
EP471651
WO 94/20526
WO 95/20603
WO97/05161
U.S. Pat. No. 4,450,105
U.S. Pat. No. 5,106,948
U.S. Pat. No. 5,169,841
WO 96/25427
U.S. Pat. No. 5,288,707
WO 96/20698
WO 01/02424

The amino acid sequence (R)-Phe-Pro-Arg, imitating amino acid sequences of fibrinogen, was at one time considered the best sequence for thrombin inhibitors. This sequence formed tight-binding inhibitors of thrombin, e.g. Ac—(R)-Phe-Pro-boroArg (DUP 714), having Ki values in the picomolar range (Kettner et al, J. Biol. Chem. 265: 18289-18297, 1990; EP-A-293,881).

The replacement of the P2 Pro residue of borotripeptide thrombin inhibitors by an N-substituted glycine is described in Fevig J M et al Bioorg. Med. Chem. 8: 301-306 and Rupin A et al Thromb. Haemost. 78(4):1221-1227, 1997. See also U.S. Pat. No. 5,585,360 (de Nanteuil et al).

Matteson D S Chem. Rev. 89: 1535-1551, 1989 reviews the use of α-halo boronic esters as intermediates for the synthesis of inter alia amino boronic acids and their derivatives. Matteson describes the use of pinacol boronic esters in non-chiral synthesis and the use of pinanediol boronic esters for chiral control, including in the synthesis of amino and amido boronate esters.

Adams et al, U.S. Pat. No. (1998), U.S. Pat. No. 6,066,730 (2000), U.S. Pat. No. 6,083,903 (2000) and equivalent, and U.S. Pat. No. 6,297,217 (2001) describe peptide boronic ester and acid compounds useful as proteasome inhibitors. These documents also describe the use of boronic ester and acid compounds to reduce the rate of muscle protein degradation, to reduce the activity of NF-κB in a cell, to reduce the rate of degradation of p53 protein in a cell, to inhibit cyclin degradation in a cell, to inhibit the growth of a cancer cell, to inhibit antigen presentation in a cell, to inhibit NF-κB dependent cell adhesion, and to inhibit HIV replication. Brand et al, WO 98/35691, teaches that proteasome inhibitors, including boronic acid compounds, are useful for treating infarcts such as occur during stroke or myocardial infarction. Elliott et al, WO 99/15183, teaches that proteasome inhibitors are useful for treating inflammatory and autoimmune diseases.

Unfortunately, organoboronic acids can be relatively difficult to obtain in analytically pure form. Thus, alkylboronic acids and their boroxines are often air-sensitive. Korcek et al, J. Chem. Soc. Perkin Trans. 2:242, 1972, teaches that butylboronic acid is readily oxidized by air to generate 1-butanol and boric acid.

It is known that derivatisation of boronic acids as cyclic esters provides oxidation resistance. For example, Martichonok V et al J. Am. Chem. Soc. 118: 950-958, 1996 state that diethanolamine derivatisation provides protection against possible boronic acid oxidation. U.S. Pat. No. 5,681,978 (Matteson D S et al) teaches that 1,2-diols and 1,3 diols, for example pinacol, form stable cyclic boronic esters that are not easily oxidised.

Wu et al, J. Pharm. Sci., 89:758-765, 2000, discuss the stability of the compound N-(2-pyrazine) carbonyl-phenylalanine-leucine boronic acid (LDP-341, also known as bortezomib), an anti-cancer agent. It is described how “during an effort to formulate [LDP-341] for parenteral administration, the compound showed erratic stability behaviour”. The degradation pathways were investigated and it was concluded that the degradation was oxidative, the initial oxidation being attributed to peroxides or molecular oxygen and its radicals.

WO 02/059131 discloses boronic acid products which are described as stable. In particular, these products are certain boropeptides and/or boropeptidomimetics in which the boronic acid group has been derivatised with a sugar.

Neutral P1 Residue Boropeptide Thrombin Inhibitors

Claeson et al (U.S. Pat. No. 5,574,014 and others) and Kakkar et al (WO 92/07869 and family members including U.S. Pat. No. 5,648,338) disclose lipophilic thrombin inhibitors having a neutral (uncharged) C-terminal (P1) side chain, for example an alkoxyalkyl side chain.

The Claeson et al and Kakkar et al patent families disclose boronate esters containing the amino acid sequence D-Phe-Pro-BoroMpg [(R)-Phe-Pro-BoroMpg], which are highly specific inhibitors of thrombin. Of these compounds may be mentioned in particular Cbz-(R)-Phe-Pro-BoroMpg-OPinacol (also known as TRI 50b). The corresponding free boronic acid is known as TRI 50c. For further information relating to TRI 50b and related compounds, the reader is referred to the following documents:
Elgendy S et al., in The Design of Synthetic Inhibitors of Thrombin, Claeson G et al Eds, Advances in Experimental Medicine, 340:173-178, 1993.
Claeson G et al, Biochem J. 290:309-312, 1993
Tapparelli C et al, J Biol Chem, 268:4734-4741, 1993
Claeson G, in The Design of Synthetic Inhibitors of Thrombin, Claeson G et al Eds, Advances in Experimental Medicine, 340:83-91, 1993
Phillip et al, in The Design of Synthetic Inhibitors of Thrombin, Claeson G et al Eds, Advances in Experimental Medicine, 340:67-77, 1993
Tapparelli C et al, Trends Pharmacol. Sci. 14:366-376, 1993
Claeson G, Blood Coagulation and Fibrinolysis 5:411-436, 1994
Elgendy et al, Tetrahedron 50:3803-3812, 1994
Deadman J et al, J. Enzyme Inhibition 9:29-41, 1995
Deadman J et al, J. Medicinal Chemistry 38:1511-1522, 1995.

TRI 50b is considered to be a prodrug for TRI 50c, which is the active principal in vivo. The tripeptide sequence of TRI 50c has three chiral centres. The Phe residue is considered to be of (R)-configuration and the Pro residue of natural (S)-configuration, at least in compounds with commercially useful inhibitor activity; the Mpg residue is believed to be of (R)-configuration in isomers with commercially useful inhibitor activity. Thus, the active, or most active, TRI 50c stereoisomer is considered to be of R,S,R configuration and may be represented as: 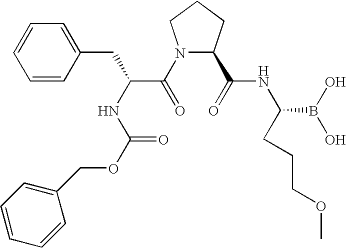
(R,S,R)-TRI 50c Cbz-(R)-Phe-(S)-Pro-(R)-Mpg-B(OH)₂

PCT/GB03/03897, and also U.S. Ser. No. 10/659,178 and EP-A-1396270, disclose pharmaceutically acceptable base addition salts of boronic acids which have a neutral aminoboronic acid residue capable of binding to the thrombin S1 subsite linked through a peptide linkage to a hydrophobic moiety capable of binding to the thrombin S2 and S3 subsites. In a first embodiment, there is disclosed a pharmaceutically acceptable base addition salt of a boronic acid of, for example, formula (A): 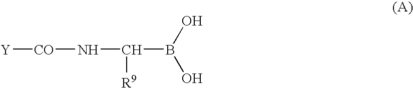
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ-W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms. TRI 50c is an exemplary acid.

Also disclosed are oral formulations of such salts.

The salts are described as being of relative stability to hydrolysis and deboronation.

PCT/GB03/03887, and also U.S. Ser. No. 10/659,179 and EP-A-1396269, disclose salts of a pharmaceutically acceptable multivalent (at least divalent) metal and an organoboronic acid drug. Such salts are described as having an improved level of stability which cannot be explained or predicted on the basis of known chemistry, and as being indicated to have unexpectedly high and consistent oral bioavailability not susceptible of explanation on the basis of known mechanisms. The oral formulations of such salts are therefore also disclosed.

One particular class of salts comprises those wherein the organoboronic acid comprises a boropeptide or boropeptidomimetic. Boropeptide drugs which may beneficially be prepared as salts include without limitation those of the formula X-(aa)ₙ-B(OH)₂, where X is H or an amino-protecting group, n is 2, 3 or 4, (especially 2 or 3) and each aa is independently a hydrophobic amino acid, whether natural or unnatural. In one class of multivalent metal salts, the organoboronic acid is of formula (A) above. TRI 50c is an exemplary acid.

PCT/GB03/03883, and also U.S. Ser. No. 10/658,971 and EP-A-1400245, disclose and claim inter alia parenteral pharmaceutical formulations that include a pharmaceutically acceptable base addition salt of a boronic acid of, for example, formula (A) above. Again, TRI 50c is an exemplary acid. Such salts are described as having an improved level of stability which cannot be explained or predicted on the basis of known chemistry.

In one aspect, the present disclosure relates, at least in embodiments, to organoboronic acid compounds designed to have an extended in vivo half life. In another aspect, the disclosure relates, at least in embodiments, to thrombin-inhibitory products which, at least in broad terms, maintain or improve the potency or specificity, or both, of such products whose active principle is TRI 50c.

In further aspects, disclosed compounds may, at least in embodiments, broadly maintain or enhance bioavailability. Other properties of disclosed compounds which may lend the compounds pharmaceutical usefulness may include storage stability or ease of formulation, for example.

### BRIEF SUMMARY OF THE DISCLOSURE

The disclosure concerns novel organoboronic acid thrombin inhibitors and the salts and prodrugs thereof (which prodrugs may themselves be in the form of salts).

In one aspect, the disclosure relates to boronic acids which have a neutral aminoboronic acid residue capable of binding to the thrombin S1 subsite linked to a hydrophobic moiety capable of binding to the thrombin S2 and S3 subsites. It also relates to salts and prodrugs of such acids. In a first embodiment, there is disclosed a boronic acid of, for example, formula (I): 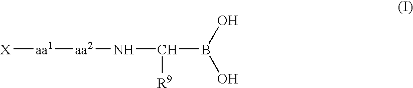
wherein
X is H (to form NH₂) or an amino-protecting group;
aa¹ is an amino acid residue having a side chain selected from formula (A) and (B):
—(CO)ₐ—(CH₂)_{b}-D_{c}-(CH₂)_{d}-E (A)
—(CO)ₐ—(CH₂)_{b}-D_{c}-Cₑ(E¹)(E²)(E³) (B)
wherein
a is 0 or 1;
e is 1;
b and d are independently 0 or an integer such that (b+d) is from 0 to 5 or, as the case may be, (b+e) is from 1 to 5;
c is 0 or 1;
D is O or S;
E is a saturated or unsaturated cyclic hydrocarbyl group which normally contains up to 14 members; and
E¹, E² and E³ are each independently selected from the group consisting of 5-6 membered hydrocarbyl rings, or one of E¹, E² and E³ is hydrogen and the other two are a said hydrocarbyl ring,
and wherein E, E¹, E² and E³ are halogenated, a particular halogen being fluorine;
   aa² is a residue of an amino carboxylic acid which binds to the thrombin S2 subsite; and
   R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ-W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms.

aa¹ may be a fluorinated Phe or Dpa residue.

The disclosure includes not only the acids as such but also their acid addition salts, base addition salts, prodrugs and prodrug salts.

Thus, the compounds are described in more detail below with particular reference to base addition salts. However, the products, methods and uses disclosed herein are not limited to the salts disclosed below but may use any boronic acid disclosed herein, or any salt or prodrug thereof (the prodrugs themselves optionally being in the form of a salt of a prodrug). It goes without saying that the salts, like the prodrugs, are pharmaceutically acceptable. In general terms, prodrugs may be boronic acid derivatives capable of hydrolysing to release the free boronic acid. As prodrugs may be mentioned esters, e.g. with a residue of an alkanol, e.g. a C₁-C₄ alkanol such as methanol or ethanol, for example. Also to be mentioned are cyclic derivatives, in which the two available valencies of the boron (corresponding to the bonds to the two —OH groups of the free acid) are bonded to respective ends of a chain of atoms, i.e. the boron becomes part of a ring. Such cyclic derivatives may be represented as below in the case of acids of Formula (I), modified mutatis mutandis for acids of other formulae disclosed herein: 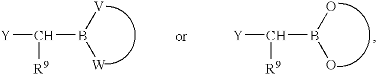
where V and W are heteroatoms (e.g. selected independently from N, O and S) and the arcuate line represents a linear or branched chain of atoms, the length of the chain between the two bonds from the boron is not critical but may be 4, 5 or 6 in some cases. As described, the chain terminated at both ends by the boron (the ring-forming chain) may be linear or branched, e.g., it may have one or more side branches; where there are multiple side branches, at least some of them may join together to form a ring, as in the case of pinanediol esters, for example.

Particular cyclic derivatives, therefore, are cyclic esters formed by diols. The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexyl-ethanediol.

The prodrug may be a sugar derivative as described in WO 02/059131 and equivalent U.S. Pat. No. 6,699,835 (see above). Thus, the boronate group may be esterified with a sugar such as a monosaccharide or a disaccharide, for example. The sugar may be a reduced sugar, e.g. manitol or sorbitol: it may be an individual sugar or class of sugars taught in WO 02/059131. The boronic acid, sugar (or other diol) and water may be combined and then lyophilised, for example as taught in WO 02/059131.

Salts may be acid addition salts or base addition salts and are, of course, pharmaceutically acceptable.

The disclosed thrombin inhibitors may contain hydrophobic amino acids, and this class of amino acids includes those whose side chain is hydrocarbyl, hydrocarbyl containing an in-chain oxygen and/or linked to the remainder of the molecule by an in-chain oxygen or heteroaryl, or any of the aforesaid groups when substituted by hydroxy, halogen or trifluoromethyl. Representative hydrophobic side chains include alkyl, alkoxyalkyl, either of the aforesaid when substituted by at least one aryl or heteroaryl, aryl, heteroaryl, aryl substituted by at least one alkyl and heteroaryl substituted by at least one alkyl. Proline and other imino acids which are ring-substituted by nothing or by one of the moieties listed in the previous sentence are also hydrophobic.

Some hydrophobic side chains contain from 1 to 20 carbon atoms, e.g. non-cyclic moieties having 1, 2, 3 or 4 carbon atoms. Side chains comprising a cyclic group typically but not necessarily contain from 5 to 13 ring members and in many cases are phenyl or alkyl substituted by one or two phenyls.

Included are inhibitors which contain hydrophobic non-peptide moieties, which are typically based on moieties which may form a side chain of a hydrophobic amino acid, as described above.

Hydrophobic compounds may contain, for example, one amino group and/or one acid group (e.g.—COOH, —B(OH)₂). Generally, they do not contain multiple polar groups of any one type.

The disclosure comprises hydrophobic boronic acid inhibitors of thrombin, and therefore includes peptide boronic acids which have a partition coefficient between 1-n-octanol and water expressed as log P of greater than 1.0 at physiological pH and 25° C. Some peptide boronic acids useful in the invention have a partition coefficient of at least 1.5. A class of hydrophobic peptide boronic acids useful in the invention has a partition coefficient of no more than 5.

Some sub-classes of hydrophobic organoboronic acids are those described by Formulae (I) and (III) below, under the heading “Detailed Description of Several Examples”.

Also disclosed as another embodiment is a peptide boronic acid of formula (II): 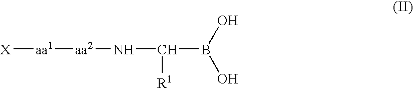
where:
X is a moiety bonded to the N-terminal amino group and may be H to form NH₂. The identity of X is not critical but may be a particular X moiety described above. In one example there may be mentioned benzyloxycarbonyl.
R¹ is a group of the formula —(CH₂)ₘ-W, where m is 2, 3 or 4 and W is —OH, —OMe, —OEt or halogen (F, Cl, Br or I).
aa¹ is a residue of an amino acid having a C₁-C₅ alkyl substituted by one or two moieties selected from fluorophenyl and fluorocyclohexyl, the fluorophenyl and fluorocyclohexyl moieties often being substituted by fluorine at the 4-position and optionally at one or more additional positions.

As previously described, the acid may be in the form of a salt (acid or base addition salt), prodrug or salt of a prodrug.

aa² is a residue of an imino acid having from 4 to 6 ring members. Alternatively, aa² is a residue of Gly N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl. Other aa² moieties are subsequently described.

There is a debate in the literature as to whether boronates in aqueous solution form the ‘trigonal’ B(OH)₂ or ‘tetrahedral’ B(OH)₃⁻ boron species, but NMR evidence seems to indicate that at a pH below the first pKa of the boronic acid the main boron species is the neutral B(OH)₂. In the duodenum the pH is likely to be between 6 and 7, so the trigonal species is likely to be predominant here. In any event, the symbol —B(OH)₂ includes tetrahedral as well as trigonal boron species, and throughout this specification symbols indicating trigonal boron species embrace also tetrahedral species. The symbol may further include boronic groups in anhydride form.

The boronic acids and their derivatives (salts, prodrugs, prodrug salts) may be in the form of solvates, particularly hydrates.

The base addition salts may comprise, or consist essentially of, acid salts in which the boronic acid is singly deprotonated. The disclosure therefore includes products having a metal/boronate stoichiometry consistent with the boronate groups in the product predominantly (more than 50 mol %) carrying a single negative charge.

The boronic acids and their derivatives may be in isolated form. The compounds may have a purity, e.g. as determined by the method of Example 9, of at least about 90%, e.g. of greater than or equal to about 95%. In the case of pharmaceutical formulations, such compound forms may be combined with pharmaceutically acceptable diluents, excipients or carriers.

Pharmaceutical formulations of the compounds are also provided herein. There are provided parenteral formulations, e.g. comprising the compounds in the solid phase, for example particulate salts or sugar esters for reconstitution as aqueous solutions prior to administration by injection or infusion. Such reconstituted solutions are also included in the disclosure. There are also provided oral preparations, e.g. tablets, capsules, powders or granules.

The disclosure also includes a pharmaceutical formulation adapted to be injected or infused into the blood of a patient, whether intravenously or in an extracorporeal blood circuit, comprising an aqueous solution of a compound selected from the group consisting of boronic acids as disclosed herein, and pharmaceutically acceptable salts, prodrugs and pharmaceutically acceptable prodrug salts of such acids. The compound may be a pharmaceutically acceptable multivalent metal salt of the boronic acid; it may be a sodium salt; it may be a reaction product of the boronic acid with an organic nitrogen containing compound having a pKb of 7 or more; it may be a reaction product of the boronic acid with an amino sugar.

According to a further aspect of the present disclosure, there is provided a method of treatment of a condition where anti-thrombotic activity is required which method comprises administration of a therapeutically effective amount of a boronic acid disclosed herein, or a salt, prodrug or prodrug salt thereof, to a person suffering from, or at risk of suffering from, such a condition.

The disclosure includes methods for preparing the described compounds.

The base addition salts described herein include products obtainable by (having the characteristics of a product obtained by) reaction of the boronic acid with a strong base and the term “salt” herein is to be understood accordingly. The term “salt” in relation to the disclosed products, therefore, does not necessarily imply that the products contain discrete cations and anions and is to be understood as embracing products which are obtainable using a reaction of a boronic acid and a base. The disclosure embraces products which, to a greater or lesser extent, are in the form of a coordination compound. The disclosure thus provides also products obtainable by (having the characteristics of a product obtained by) reaction of a disclosed boronic acid with a strong base a well as the therapeutic, including prophylactic, use of such products.

The disclosure therefore includes a method for preparing a product, the method comprising contacting an organoboronic acid of the disclosure, e.g. of formula (I), with a pharmaceutically acceptable base. Suitably, the pharmaceutically acceptable base can provide cations having a valency n and the base is added in such an amount that the organoboronic acid and the cations are in a stoichiometry of n:1 (organoboronic acid:cations). For example, a base containing calcium can provide divalent cations (and of course does so when the method is performed), and half as many moles of calcium are therefore contacted with the boronic acid as the number of moles of the acid. The method may further comprise formulating the product into a pharmaceutical formulation, e.g. intravenous (including for introduction into an extracorporeal blood circuit) or oral. The organoboronic acid may be TRI 50f (see below).

The present disclosure is not limited as to the method of preparation of the base addition salts, provided that they contain a boronate species derived from a disclosed boronic acid and a counter-ion. Such boronate species may be boronate anions in any equilibrium form thereof. The term “equilibrium form” refers to differing forms of the same compounds which may be represented in an equilibrium equation (e.g. boronic acid in equilibrium with a boronic anhydride and in equilibrium with different boronate ions). Boronates in the solid phase may form anhydrides and the disclosed boronate salts when in the solid phase may comprise boronate anhydrides, as a boronic equilibrium species. It is not required that the salts be prepared by reaction of a base containing the counter-ion and the boronic acid (I). Further, the disclosure includes salt products which might be regarded as indirectly prepared by such an acid/base reaction as well as salts obtainable by (having the characteristics of products obtained by) such indirect preparation. As examples of possibly indirect preparation may be mentioned processes in which, after initial recovery of the salt, it is purified and/or treated to modify its physicochemical properties, for example to modify solid form or hydrate form, or both.

The invention includes a medicament comprising a salt, sugar ester or other soluble derivative of a boronic acid which is a selective thrombin inhibitor and has a neutral aminoboronic acid residue capable of binding to the thrombin S1 subsite linked to a hydrophobic moiety capable of binding to the thrombin S2 and S3 subsites, the hydrophobic moiety comprising a fluorinated aromatic ring in its S3-binding part and the salt comprising a cation having a valency n and having an observed stoichiometry consistent with a notional stoichiometry (boronic acid:cation) of n:1.

In some embodiments, the cations of the base addition salts are monovalent.

In some embodiments the disclosed compounds (e.g. free boronic acids or salts) comprise anhydride species; in others they are essentially free of anhydride species.

Further aspects and embodiments of the disclosure are set forth in the following description and claims.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

Data indicate that the stability (resistance to deboronation) of organoboronic acids may be increased by providing them in the form of salts, e.g. metal salts. In single experiments, the ammonium salt of TRI 50c appeared to decompose on drying to yield ammonia, whilst the choline salt demonstrated rapid decomposition to a deboronated impurity. Although experiments have not been conducted to reproduce these unrepeated observations, there is provided a sub-class of the presently disclosed salts in which the ammonium and choline salts are excluded.

### DETAILED DESCRIPTION OF SEVERAL EXAMPLES

Glossary

The following terms and abbreviations are used in this specification:

The expression “acid salt” as applied to a base addition salt of a boronic acid refers to salts of which a single —OH group of the trigonally-represented acid group —B(OH)₂ is deprotonated. Thus salts wherein the boronate group carries a single negative charge and may be represented as —B(OH)(O⁻) or as [—B(OH)₃]⁻ are acid salts. The expression encompasses salts of a cation having a valency n wherein the molar ratio of boronic acid to cation is approximately n to 1. In practical terms, the observed stoichiometry is unlikely to be exactly n:1 but will be consistent with a notional n:1 stoichiometry. For example, the observed mass of the cation might vary from the calculated mass for a n:1 stoichiometry by no more than about 10%, e.g. no more than about 7.5%; in some cases an observed mass of a cation might vary from the calculated mass by no more than about 1%. Calculated masses are suitably based on the trigonal form of the boronate. (At an atomic level, a salt stoichiometrically consistent with being an acid salt might contain boronates in a mix of protonation states, whose average approximates to single deprotonation and such “mixed” salts are included in the term “acid salt”). Examples of acid salts are monosodium salts and hemicalcium salts.

α-Aminoboronic acid or Boro(aa) refers to an amino acid in which the CO₂ group has been replaced by BO₂.

The term “amino-group protecting moiety” refers to any group used to derivatise an amino group, especially an N-terminal amino group of a peptide or amino acid. Such groups include, without limitation, alkyl, acyl, alkoxycarbonyl, aminocarbonyl, and sulfonyl moieties. However, the term “amino-group protecting moiety” is not intended to be limited to those particular protecting groups that are commonly employed in organic synthesis, nor is it intended to be limited to groups that are readily cleavable.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The expression “thrombin inhibitor” refers to a product which, within the scope of sound pharmacological judgement, is potentially or actually pharmaceutically useful as an inhibitor of thrombin, and includes reference to substance which comprises a pharmaceutically active species and is described, promoted or authorised as a thrombin inhibitor. Such thrombin inhibitors may be selective, that is they are regarded, within the scope of sound pharmacological judgement, as selective towards thrombin in contrast to other proteases; the term “selective thrombin inhibitor” includes reference to substance which comprises a pharmaceutically active species and is described, promoted or authorised as a selective thrombin inhibitor.

The term “heteroaryl” refers to a ring system which has at least one (e.g. 1, 2 or 3) in-ring heteroatoms and has a conjugated in-ring double bond system. The term “heteroatom” includes oxygen, sulfur and nitrogen, of which sulfur is sometimes less preferred.

“Natural amino acid” means an L-amino acid (or residue thereof) selected from the following group of neutral (hydrophobic or polar), positively charged and negatively charged amino acids:

Hydrophobic Amino Acids
A=Ala=alanine
V=Val=valine
I=Ile=isoleucine
L=Leu=leucine
M=Met=methionine
F=Phe=phenylalanine
P=Pro=proline
W=Trp=tryptophan

Polar (Neutral or Uncharged) Amino Acids
N=Asn=asparagine
C=Cys=cysteine
Q=Gln=glutamine
G=Gly=glycine
S=Ser=serine
T=Thr=threonine
Y=Tyr=tyrosine

Positively Charged (Basic) Amino Acids
R=Arg=arginine
H=His=histidine
K=Lys=lysine

Negatively Charged Amino Acids
D=Asp=aspartic acid
E=Glu=glutamic acid.
   ACN=acetonitrile
   Acid addition salt=a salt which is prepared from addition of an inorganic acid or an organic acid to a free base (e.g. an amino group, as for example an N-terminal amino group of a peptide).
   Active principle=Chemical component of a plant or compound that has a therapeutic effect, e.g. in the case of a salt or prodrug of a boronic acid the active principle is the boronic acid (in this context, corresponding boronate ions may be considered as equivalent to the acid)
   Amino acid=α-amino acid
   Base addition salt=a salt which is prepared from addition of an inorganic base or an organic base to a free acid (in this case the boronic acid).
   CABG=coronary artery bypass graft(ing)
   Cbz=benzyloxycarbonyl
   Cha=cyclohexylalanine (a hydrophobic unnatural amino acid)
   Charged (as applied to drugs or fragments of drug molecules, e.g. amino acid residues)=carrying a charge at physiological pH, as in the case of an amino, amidino or carboxy group
   CIHD=chronic intermittent haemodialysis
   CPB=cardiopulmonary bypass
   Dcha=dicyclohexylalanine (a hydrophobic unnatural amino acid)
   Dpa=diphenylalanine (a hydrophobic unnatural amino acid)
   Drug=a pharmaceutically useful substance, whether the active in vivo principle or a prodrug
   i.v.=intravenous
   Mpg=3-methoxypropylglycine (a hydrophobic unnatural amino acid)
   Multivalent=valency of at least two, for example two or three
   Neutral (as applied to drugs or fragments of drug molecules, e.g. amino acid residues)=uncharged=not carrying a charge at physiological pH
   Pinac=Pinacol=2,3-dimethyl-2,3-butanediol
   Pinanediol=2,3-pinanediol=2,6,6-trimethylbicyclo[3.1.1]heptane-2,3-diol
   Pip=pipecolinic acid
   Room temperature=25° C.±2° C.
   s.c.=subcutaneous
   Strong base=a base having a sufficiently high pKb to react with a boronic acid. Suitably such bases have a pKb of 7 or more, e.g. 7.5 or more, for example about 8 or more
   THF=tetrahydrofuran
   Thr=thrombin
   TRI 50f=the analogue of TRI 50c in which the Phe residue is 4-fluorinated
   Novel Product—The Compounds

The disclosure relates to boronic acids and their salts, prodrugs and prodrug salts. It relates to the pharmaceutical use of these compounds.

The boronic acids of the disclosure comprise in one aspect boronic acids which have a neutral aminoboronic acid residue capable of binding to the thrombin S1 subsite linked to a hydrophobic moiety capable of binding to the thrombin S2 subsite, which is linked in turn to a moiety which is capable of binding to the thrombin S3 subsite and comprises a fluorinated ring, for example a 6-membered ring which is 4-fluorinated. The substituent on the amino group, if any, is generally an optionally substituted hydrocarbyl group, particularly an alkyl group or substituted alkyl group. The alkyl group may be halogenated, particularly fluorinated. The disclosure includes also acids of formula (I): 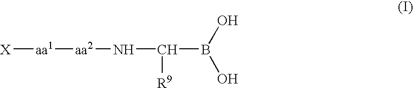
wherein
X is H (to form NH₂) or an amino-protecting group;
aa¹ is an amino acid residue having a side chain selected from formula (A) and (B):
—(CO)ₐ—(CH₂)_{b}-D_{c}-(CH₂)_{d}-E (A)
—(CO)ₐ—(CH₂)_{b}-D_{c}-Cₑ(E¹)(E²)(E³) (B)
wherein
a is 0 or 1;
e is 1;
b and d are independently 0 or an integer such that (b+d) is from 0 to 5 or, as the case may be, (b+e) is from 1 to 5;
c is 0 or 1;
D is O or S;
E is a saturated or unsaturated cyclic hydrocarbyl group which normally contains up to 14 members; and
E¹, E² and E³ are each independently selected from the group consisting of 5-6 membered hydrocarbyl rings, or one of E¹, E² and E³ is hydrogen and the other two are a said hydrocarbyl ring,
and wherein E, E¹, E² and E³ are halogenated, a particular halogen being fluorine;
   aa² is a residue of an amino acid which binds to the thrombin S2 subsite; and
   R⁹ is a straight chain alkyl group interrupted by one or more ether linkages and in which the total number of oxygen and carbon atoms is 3, 4, 5 or 6 (e.g. 5) or R⁹ is —(CH₂)ₘ-W where m is from 2, 3, 4 or 5 (e.g. 4) and W is —OH or halogen (F, Cl, Br or I). As examples of straight chain alkyl interrupted by one or more ether linkages (—O—) may be mentioned alkoxyalkyl (one interruption) and alkoxyalkoxyalkyl (two interruptions). R⁹ is an alkoxyalkyl group in one subset of compounds, e.g. alkoxyalkyl containing 4 carbon atoms.

E particularly comprises a 5-6 membered ring (e.g. is 4-fluorophenyl) or an 8-14 membered fused ring system (e.g. is 4-fluoronaphthyl).

In many compounds, aa¹ has a side chain in which a is 0. If a is 1, c may be 0. Commonly, a and c are both 0. In particular examples, (a+b+c+d) and (a+b+c+e) are no more than 4 and are more especially 1, 2 or 3. (a+b+c+d) may be 0.

In one class of compounds, a and c are both 0 and (a+b+c+d)) and (a+b+c+e) are 1, 2 or 3. In particular, aa¹ has a side chain which is one of:
—CH₂-E (A′)
—C(E¹)(E²)(E³) (B′)

In one class of compounds, E, E¹, E² and E³ are each independently phenyl fluorinated at least the 4-position or fluorinated cyclohexyl, the fluorinated cyclohexyl typically being fluorinated at least the 4-position; in variants of this class one of E¹, E² and E³ is H. In this class of compounds, the number of fluorinated carbon atoms in the phenyl groups, in addition to the 4-carbon, may be 1, 2, 3 or 4. Some exemplary E, E¹, E² and E³ groups are:

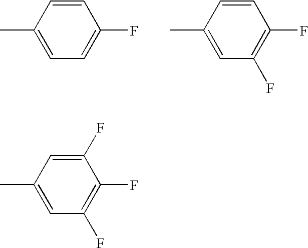

Cyclohexyl may be fluorinated as described above in relation to phenyl. As previously stated, particular side chains are those in which a and c are both 0 and (a+b+c+d) and/or (a+b+c+e) are 1.

Particular boronic acids have aa¹ side chains selected from: 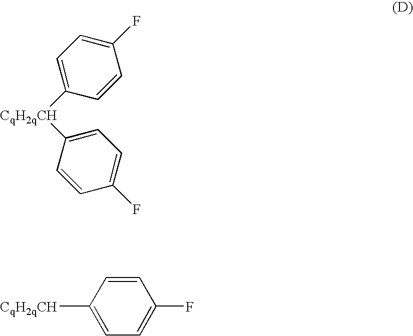
wherein q is from 0 to 5, e.g. is 0, 1 or 2, especially 1 (in which latter case the side chains are fluorinated variants of Phe and Dpa). Of course, the phenyl groups may be additionally fluorinated as described above (e.g. 3,4 or 3,4,5 fluorinated); again the same applies to the corresponding compounds in which phenyl is replaced by cyclohexyl.

In one class of compounds, the side chain of the P2 (S2-binding) amino acid is a moiety other than hydrogen selected from a group of formula A or B:
—(CO)ₐ—(CH₂)_{b}-D_{c}-(CH₂)_{d}-E (A)
—(CO)ₐ—(CH₂)_{b}-D_{c}-Cₑ(E¹)(E²)(E³) (B)
wherein
a is 0 or 1;
e is 1;
b and d are independently 0 or an integer such that (b+d) is from 0 to 5 or, as the case may be, (b+e) is from 1 to 5;
c is 0 or 1;
D is O or S;
E is H, C₁-C₆ alkyl, or a saturated or unsaturated cyclic group which normally contains up to 14 members and particularly is a 5-6 membered ring (e.g. phenyl) or an 8-14 membered fused ring system (e.g. naphthyl), which alkyl or cyclic group is optionally substituted by up to 3 groups (e.g. 1 group) independently selected from C₁-C₆ trialkylsilyl, —CN, —R¹³, —R¹²OR¹³, —R¹²COR¹³, —R¹²CO₂R¹³ and —R¹²O₂CR¹³, wherein R¹² is —(CH₂)_{f}— and R¹³ is —(CH₂)_{g}H or by a moiety whose non-hydrogen atoms consist of carbon atoms and in-ring heteroatoms and number from 5 to 14 and which contains a ring system (e.g. an aryl group) and optionally an alkyl and/or alkylene group, wherein f and g are each independently from 0 to 10, g particularly being at least 1 (although —OH may also be mentioned as a substituent), provided that (f+g) does not exceed 10, more particularly does not exceed 6 and most particularly is 1, 2, 3 or 4, and provided that there is only a single substituent if the substituent is a said moiety containing a ring system, or E is C₁-C₆ trialkylsilyl; and E¹, E² and E³ are each independently selected from —R¹⁵ and -J-R¹⁵, where J is a 5-6 membered ring and R¹⁵ is selected from C₁-C₆ trialkylsilyl, —CN, —R¹³, —R¹²OR¹³, —R¹²COR¹³, —R¹²CO₂R¹³, —R¹²O₂CR¹³, and one or two halogens (e.g. in the latter case to form a -J-R¹⁵ moiety which is dichlorophenyl), where R¹² and R¹³ are, respectively, an R¹² moiety and an R¹³ moiety as defined above (in some acids where E¹, E² and E³ contain an R¹³ group, g is 0 or 1);
in which moiety of Formula (A) or (B) any ring is carbocyclic or aromatic, or both, and any one or more hydrogen atoms bonded to a carbon atom is optionally replaced by halogen, especially F.

In certain examples, a is 0. If a is 1, c may be 0. In particular examples, (a+b+c+d) and (a+b+c+e) are no more than 4 and are more especially 1, 2 or 3. (a+b+c+d) may be 0.

Exemplary groups for E, E¹, E² and E³ include aromatic rings such as phenyl, naphthyl, pyridyl, quinolinyl and furanyl, for example; non-aromatic unsaturated rings, for example cyclohexenyl; saturated rings such as cyclohexyl, for example. E may be a fused ring system containing both aromatic and non-aromatic rings, for example fluorenyl. One class of E, E¹, E² and E³ groups are aromatic (including heteroaromatic) rings, especially 6-membered aromatic rings. In some compounds, E¹ is H whilst E² and E³ are not H; in those compounds, examples of E² and E³ groups are phenyl (substituted or unsubstituted) and C₁-C₄ alkyl, e.g. methyl.

In one class of embodiments, E contains a substituent which is C₁-C₆ alkyl, (C₁-C₅ alkyl)carbonyl, carboxy C₁-C₅ alkyl, aryl (including heteroaryl), especially 5-membered or preferably 6-membered aryl (e.g. phenyl or pyridyl), or arylalkyl (e.g. arylmethyl or arylethyl where aryl may be heterocyclic and is preferably 6-membered).

In another class of embodiments, E contains a substituent which is OR¹³, wherein R¹³ can be a 6-membered ring, which may be aromatic (e.g. phenyl) or is alkyl (e.g. methyl or ethyl) substituted by such a 6-membered ring.

A class of moieties of formula A or B are those in which E is a 6-membered aromatic ring optionally substituted, particularly at the 2-position or 4-position, by —R¹³ or —OR¹³.

The disclosure includes acids in which the P3 and/or P2 side chain comprises a cyclic group in which 1 or 2 hydrogens have been replaced by halogen, e.g. F or Cl.

The disclosure includes a class of acids in which the P2 side chains of formula (A) or (B) are of the following formulae (i), (ii) or (iii): 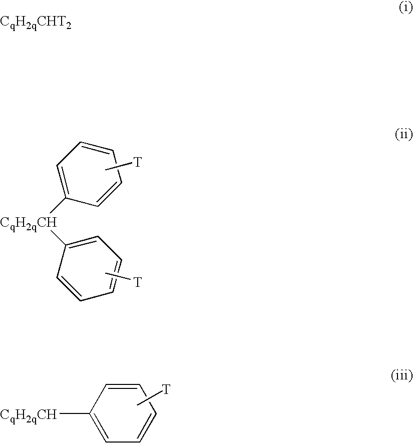
wherein q is from 0 to 5, e.g. is 0, 1 or 2, and each T is independently hydrogen, one or two halogens (e.g. F or Cl), —SiMe₃, —CN, —R¹³, —OR¹³, —COR¹³, —CO₂R¹³ or —O₂CR¹³. In some embodiments of structures (ii) and (iii), T is at the 4-position of the phenyl group(s) and is —R¹³, —OR¹³, —COR¹³, —CO₂R¹³ or —O₂CR¹³, and R¹³ is C₁-C₁₀ alkyl and more particularly C₁-C₆ alkyl. In one sub-class, T is —R¹³ or —OR¹³, for example in which f and g are each independently 0, 1, 2 or 3; in some side chains groups of this sub-class, T is —R¹²OR¹³ and R¹³ is H.

In one class of the moieties, the side chain is of formula (i) and each T is independently R¹³ or OR¹³ and R¹³ is C₁-C₄ alkyl. In some of these compounds, R¹³ is branched alkyl and in others it is straight chain. In some moieties, the number of carbon atoms is from 1 to 4.

The disclosure therefore includes medicaments comprising organoboronic acids or derivatives thereof which are thrombin inhibitors, particularly selective thrombin inhibitors, having a neutral P1 (S1-binding) moiety and a halogenated P1 moiety as disclosed herein. For more information about moieties which bind to the S3, S2 and S1 sites of thrombin, see for example Tapparelli C et al, Trends Pharmacol Sci. 14: 366-376, 1993; Sanderson P et al, Current Medicinal Chemistry, 5: 289-304, 1998; Rewinkel J et al, Current Pharmaceutical Design, 5:1043-1075, 1999; and Coburn C Exp. Opin. Ther. Patents 11(5): 721-738, 2001. The thrombin inhibitory compounds of the disclosure are not limited to those having S2 and S1 affinity groups described in the publications listed in the preceding sentence. Particular derivatives are base addition salts.

The boronic acids may have a Ki for thrombin of about 100 nM or less, e.g. about 20 nM or less.

A subset of the Formula (I) acids comprises the acids of Formula (II): 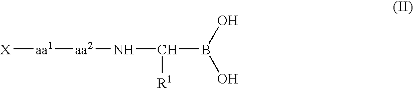
X is a moiety bonded to the N-terminal amino group and may be H to form NH₂. The identity of X is not critical but may be a particular X moiety described above. In one example there may be mentioned benzyloxycarbonyl.

In certain examples X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 (of which 0 and 1 are preferred) and R⁶ is H or a 5 to 13-membered cyclic group optionally substituted by one or more (e.g. 1, 2, 3, 4 or 5) halogens (e.g. F), for example at least at the 4-position, and/or by 1, 2 or 3 substituents selected from amino, nitro, hydroxy, a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group.

More particularly X is R⁶—(CH₂)ₚ—C(O)— or R⁶—(CH₂)ₚ—O—C(O)— and p is 0, 1 or 2; in these moieties, R⁶ may be phenyl or fluorophenyl. Said 5 to 13-membered cyclic group is often aromatic or heteroaromatic, for example is a 6-membered aromatic or heteroaromatic group. The cyclic group may be saturated, particularly it may be a cycloalkyl group, e.g. cyclohexyl, optionally substituted by one or more halogens (e.g. F). When R⁶ comprises an F-substituted 6-membered ring, the ring is typically at least 4-substituted. In many cases, the group is not substituted.

Exemplary X groups are (2-pyrazine) carbonyl, (2-pyrazine) sulfonyl and particularly benzyloxycarbonyl or benzylmethylcarbonyl.

aa¹ is as previously described. Thus, it may have a side chain which is —CH₂-E or —C(E¹)(E²)(E³), in particular where one of E¹, E² and E³ is H. More particularly, aa¹ may be 4-F-Phe or di(4-F)-Dpa. (4-F-Phe refers to Phe whose phenyl group is 4-fluorinated; di(4-F)-Dpa refers to Dpa both of whose phenyl groups are 4-fluorinated). Also to be mentioned are the corresponding analogues in which phenyl is replaced by cyclohexyl, i.e. 4-F-Cha and di(4-F)Dcha.

aa² is an imino acid residue having from 4 to 6 ring members. Alternatively, aa² is a residue of H₂N—CH₂—COOH N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

As another alternative, aa² is the β-amino acid analogue of Gly (i.e. H₂N—CH₂—CH₂—COOH) N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

The disclosure includes a class of compounds in which aa² is a residue of a β-amino acid having a 4 to 6 membered carbocyclic ring which optionally has one carbon atom replaced by a sulfur and of which the ring-forming carbon atoms include the carbon atoms α- and β- to the carboxyl group (i.e. the β-amino acid comprises a 4 to 6 membered carbocyclic ring which is 1-substituted by carboxyl and 2-substituted by amino and which may at one other position contain an S atom).

An exemplary class of products comprises those in which aa² is a residue of an imino acid of formula (IV) 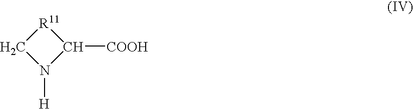
where R¹¹ is —CH₂—, —CH₂—CH₂—, —CH═CH—, —S—CH₂— or —CH₂—CH₂—CH₂—, which group when the ring is 5 or 6-membered is optionally substituted at one or more —CH₂— groups by from 1 to 3 C₁-C₃ alkyl groups, for example to form the R¹¹ group —S—C(CH₃)₂—. Of these imino acids, azetidine-2-carboxylic acid, especially (s)-azetidine-2-carboxylic acid, and more particularly proline-C(O)OH are illustrative.

Also to be mentioned as aa² are β-amino acids of formula β: 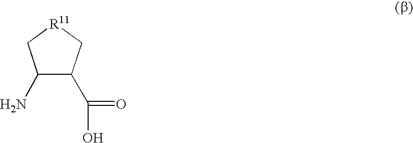
wherein R¹¹ is as previously defined.

In embodiments, aa² is a residue of an N-substituted imino acid or N-substituted β-amino acid.

It will be appreciated from the above that a very preferred class of products consists of those in which aa¹-aa² is 4-F-Phe-Pro. In another preferred class, aa¹-aa² is di(4-F)-Dpa-Pro. In other products, aa¹-aa² is Cha-Pro or Dcha-Pro. Of course, also included are corresponding product classes in which Pro is replaced by (s)-azetidine-2-carboxylic acid.

R⁹ is as defined previously and may be a moiety R¹ of the formula —(CH₂)ₛ-Z. Integer s is 2, 3 or 4 and W is —OH, —OMe, —OEt or halogen (F, Cl, I or, preferably, Br). Particularly illustrative Z groups are —OMe and —OEt, especially —OMe. In certain examples s is 3 for all Z groups and, indeed, for all compounds of the disclosure. Particular R¹ groups are 2-bromoethyl, 2-chloroethyl, 2-methoxyethyl, 4-bromobutyl, 4-chlorobutyl, 4-methoxybutyl and, especially, 3-bromopropyl, 3-chloropropyl and 3-methoxypropyl. Most preferably, R¹ is 3-methoxypropyl. 2-Ethoxyethyl is another preferred R¹ group.

Accordingly, a specific class of compounds consists of those of acids of the formula X-4-F-Phe-Pro-Mpg-B(OH)₂, especially Cbz-4-F-Phe-Pro-Mpg-B(OH)₂; also included are analogues of these compounds in which Mpg is replaced by a residue with another of the R¹ groups and/or 4-F-Phe is replaced by di(4-F)-Dpa or another disclosed aa¹ residue. Also included are compounds in which Cbz is replaced by benzylmethylcarbonyl (Ph-Et-CO—).

The aa¹ moiety of the product is preferably of R configuration. The aa² moiety is preferably of (S)-configuration. Particularly preferred products have aa¹ of (R)-configuration and aa² of (S)-configuration. The chiral centre —NH—CH(R¹)—B— is preferably of (R)-configuration. It is considered that commercial formulations will have the chiral centres in (R,S,R) arrangement, as for example in the case of salts of Cbz-4-F-Phe-Pro-boroMpg-OH (TRI 50f):

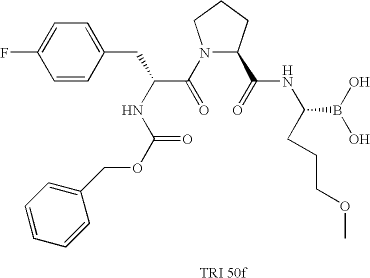

The disclosure includes Cbz-(R)-4-F-Phe-(S)-Pro-(R)-boroMpg-OH and Cbz-(R)-di-(4-F)-Dpa-(S)-Pro-(R)-boroMpg-OH (and other compounds of the formula X—(R)-4-F-Phe-(S)-Pro-(R)-boroMpg-OH and X—(R)-di-(4-F)-Dpa-(S)-Pro(R)-boroMpg-OH) which for example are at least 90% pure, e.g. at least 95% pure. These compounds may be in the form of, e.g., base addition salts.

Also disclosed herein are acid addition salts, which may be formed by contacting a disclosed acid with a pharmaceutically acceptable acid, particularly a strong inorganic acid, e.g. HBr, HCl or HSO₂CH₃. The pharmaceutically acceptable acid will form a salt with an amino group, e.g. an unprotected N-terminal amino group.

In broad terms, the base addition salts described herein may be considered to correspond to reaction products of an organoboronic acid as described above with a strong base, e.g. a basic metal compound; the salts are however not limited to products resulting from such a reaction and may be obtained by alternative routes.

The base addition salts are therefore obtainable by contacting an acid of the disclosure with a strong base. The disclosure thus contemplates products (compositions of matter) having the characteristics of a reaction product of a disclosed acid and a strong base. The base is pharmaceutically acceptable.

As suitable salts may be mentioned salts of metals, e.g. of monovalent or divalent metals, and stronger organic bases, for example:

1. Alkali metal salts;

2. Divalent, e.g. alkaline earth metal, salts;

3. Group III metals;

4. Salts of strongly basic organic nitrogen-containing compounds, including:

4A. Salts of guanidines and their analogues;
4B. Salts of strongly basic amine, examples of which include (i) aminosugars and (ii) other amines.

Of the above salts, particularly illustrative are alkali metals, especially Na and Li. Also illustrative are aminosugars.

Specific salts are of the acid boronate though in practice the acid salts may contain a very small proportion of the doubly deprotonated boronate. The term “acid boronate” refers to trigonal —B(OH)₂ groups in which one of the B—OH groups is deprotonated as well as to corresponding tetrahedral groups in equilibrium therewith. Acid boronates have a stoichiometry consistent with single deprotonation.

Accordingly, the disclosure includes base addition salts of the disclosed boronic acids, for example those of Formula (I), which have an observed stoichiometry consistent with the organoboronic acid being in the form of a salt of which a single —OH group of the trigonally-represented boronyl group —B(OH)₂ is deprotonated or, in an alternative expression of the same deprotonation state, in which the boronyl group carries a single negative charge and is in a form selected from the group consisting of the following equilibrium species or a combination thereof:

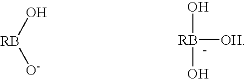

In the above formulae, R represents the organic moiety with which the boron is substituted. For example, in the case of species derived from free acids of formula (I), R is the following sub-structure found within formula (I):

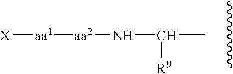

The disclosure includes products (compositions of matter) which comprise salts which may be represented by formula (V): 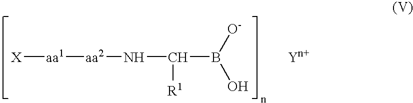
where Yⁿ⁺ is a pharmaceutically acceptable cation obtainable from a strong base, and aa¹, aa², X and R¹ are as defined above. Also included are products in which R¹ is replaced by another R⁹ group. Also included are corresponding compounds in which the peptidoboronyl group of Formula (V) is replaced by another peptidoboronyl group disclosed herein.

One class of salts have a solubility of about 10 mM or more, e.g. of at least about 20 mM, when their solubility is determined as described in the examples at a dissolution of 25 mg/ml. More particularly yet they have a solubility of least 50 mM when their solubility is determined as described in the examples at a dissolution of 50 mg/ml.

The disclosure includes salts of boronic acids (I) having an observed stoichiometry consistent with the salt being of (being representable by) the formula “(boronate⁻)ₙ cationⁿ⁺”. One class of such salts are represented by the formula:
[Cbz-(R)-Phe-(S)-Pro-(R)-Mpg-B(OH)(O⁻)]M⁺
where M⁺ represents a monovalent cation, especially an alkali metal cation. It will be understood that the above representation is a notional representation of a product whose observed stoichiometry is unlikely to be literally and exactly 1:1. In any event, a particular salt is Cbz-(R)-Phe-(S)-Pro-(R)-Mpg-B(OH)₂ monosodium salt (TGN 255). In the above formula, the trigonally-represented boronate represents, as always, boronates which are trigonal, tetrahedral or mixed trigonal/tetrahedral.

Particularly exemplary are products which comprise:

(i) species selected from (a) acids of formula (VIII): X—(R)-Phe-(S)-Pro-(R)-Mpg-B(OH)₂ where X is H or an amino-protecting group, especially Cbz, (b) boronate anions thereof, and (c) any equilibrium form of the aforegoing (e.g. an anhydride); and

(ii) ions having a valency n in combination with said species, the species and said ions having an observed stoichiometry consistent with a notional species:ion stoichiometry of n:1. In one class of salts, n is 1.

In the following part of this specification, the various possible counter-ions are considered with reference to boronic acids of the following Formula (IIIA): 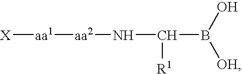
where the various symbols have the meaning ascribed to them previously. Other boronic acid drugs, for example compounds of Formula (I) or others referred to in this specification, may of course be used in place of those of Formula (I). Considering the counter-ions in turn, therefore:
1. Monovalent Metal, Especially Alkali Metal Salts

Suitable alkali metals include lithium, sodium and potassium. All of these are remarkably soluble. Lithium and sodium are illustrative because of their high solubility. The lithium and particularly sodium salts are of surprisingly high solubility in relation to potassium amongst others. Sodium is most used in many instances. Salts containing mixtures of alkali metals are contemplated by the disclosure.

The disclosure includes products comprising salts of the formula (VI) 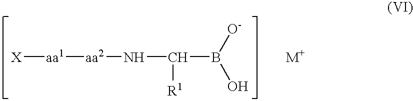
where M⁺ is an alkali metal ion and aa¹, aa², X and R¹ are as defined above, as well as salts in which both hydroxy groups of the boronate group are in salt form (preferably with another identical M⁺ group) and mixtures of such salts. Included also are products wherein R¹ is replaced by another R⁹ group.
2. Divalent, e.g. Alkaline Earth Metal (Group II Metal) Salts

One example of a divalent metal is calcium. Another suitable divalent metal is magnesium. Also contemplated is zinc. The divalent metals are usually used in a boronic acid:metal ratio of substantially 2:1, in order to achieve the preferred monovalent boronate moiety. Salts containing mixtures of divalent metals, e.g. mixtures of alkaline earth metals, are also contemplated.

Further disclosed are products (compositions of matter) which comprise salts which may be represented by the formula (VII): 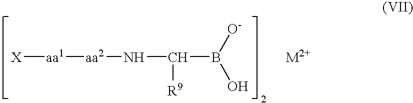
where M²⁺ is a divalent metal cation, e.g. an alkaline earth metal or zinc cation, and aa¹, aa², X and R⁹ are as defined above, as well as salts in which both hydroxy groups of the boronate group are deprotonated and mixtures of such salts. As previously indicated, the boronate may comprise a tetrahedral species.
3. Group III Metals

Suitable Group III metals include aluminium and gallium. Salts containing mixtures of Group III metals are also contemplated.

The disclosure includes products comprising salts of the formula (VIII): 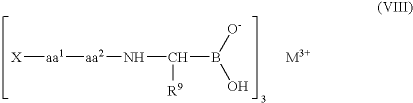
where M³⁺ is a Group III metal ion and aa¹, aa², X and R⁹ are as defined above, as well as salts in which both hydroxy groups of the boronate group are in salt form and mixtures of such salts. As previously indicated, the boronate may comprise a tetrahedral species.
4. Strongly Basic Organic Nitrogen-Containing Compounds

The disclosure includes products obtainable by (having the characteristics of a product obtained by) reaction of a peptide boronic acid as defined above and a strong organic base. Two illustrative classes of organic base are described in sections 4A and 4B below. Particularly preferred are acid salts (in which one of the two boronic —OH groups is deprotonated). Most commonly, the salts contain a single type of organic counter-ion (disregarding trace contaminants) but the disclosure contemplates salts containing mixtures of organic counter-ions; in one sub-class, the different counter-ions all fall within the section 4A family described below or, as the case may be, in the section 2B family below; in another subclass, the salts comprise a mixture of organic counter-ions which are not all from the same family (4A or 4B).

Suitable organic bases include those with a pKb of 7 or more, e.g. 7.5 or more, for example in the region of 8 or more. Bases which are less lipophilic [e.g. have at least one polar functional group (e.g. 1, 2 or 3 such groups) for example hydroxy] are favoured; thus aminosugars are one favoured class of base.

4A. Guanidines and Their Analogues

The guanidino compound (guanidine) may in principle be any soluble and pharmaceutically acceptable compound having a guanidino or a substituted guanidino group, or a substituted or unsubstituted guanidine analogue. Suitable substituents include aryl (e.g. phenyl), alkyl or alkyl interrupted by an ether or thioether linkage and, in any event, typically contain from 1 to 6 and especially 1, 2, 3, or 4 carbon atoms, as in the case of methyl or ethyl. The guanidino group may have 1, 2, 3 or 4 substituent groups but more usually has 1 or 2 substituent groups, for instance on a terminal nitrogen. One class of guanidines is monoalkylated; another class is dialkylated. As guanidine analogues may be mentioned thioguanidines and 2-amino pyridines. Compounds having unsubstituted guanidino groups, for example guanidine and arginine, form one particular class.

Salts containing mixtures of guanidines are contemplated by the disclosure.

A particular guanidino compound is L-arginine or an L-arginine analogue, for example D-arginine, or the D- or, preferably, L-isomers of homoarginine or agmatine[(4-aminobutyl)guanidine]. Less preferred arginine analogues are NG-nitro-L-arginine methyl ester, for example, and constrained guanidine analogues, particularly 2-amino pyrimidines, for example 2, 6-quinazolinediamines such as 5,6,7,8-tetrahydro-2,6-quinazolinediamine, for example. The guanidino compound may also be a peptide, for example a dipeptide, containing arginine; one such dipeptide is L-tyrosyl-L-arginine.

Some particular guanidino compounds are compounds of formula (VII): 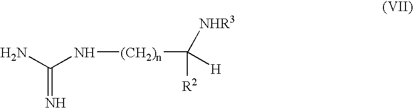
where n is from 1 to 6 and for example at least 2, e.g. 3 or more, and in many instances no more than 5. Most particularly, n is 3, 4 or 5. R² is H or carboxylate or derivatised carboxylate, for example to form an ester (e.g. a C₁-C₄ alkyl ester) or amide. R³ is H, C₁-C₄ alkyl or a residue of a natural or unnatural amino acid (e.g. tyrosine). The compounds of formula (IV) are usually of L-configuration. The compounds of formula (IV) are arginine (n=3; R²=carboxyl; R³=H) and arginine derivatives or analogues.

The disclosure includes products comprising salts of the formula (IX) 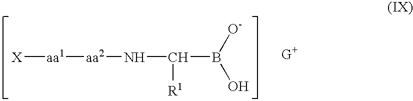
where aa¹, aa², X and R¹ are as defined previously and G⁺ is the protonated form of a pharmaceutically acceptable organic compound comprising a guanidino group or an analogue thereof, as well as salts in which both hydroxy groups of the boronate group are in salt form (preferably with another identical G⁺ group) and mixtures of such salts. Also included are products wherein R¹ is replaced by another R⁹ group.
4B. Strongly Basic Amines

The disclosure includes products obtainable by (having the characteristics of a product obtained by) reaction of a peptide boronic acid as defined above and a strong organic base which is an amine. The amine may in principle be any soluble and pharmaceutically acceptable amine.

It is envisaged that a desirable class of amine includes those having polar functional groups in addition to a single amine group, as such compounds will be more hydrophilic and thus more soluble than others. In certain salts, the or each additional functional group is hydroxy. Some amines have 1, 2, 3, 4, 5 or 6 additional functional groups, especially hydroxy groups. In one illustrative class of amines the ratio of (amino plus hydroxy groups):carbon atoms is from 1:2 to 1:1, the latter ratio being particularly preferred. These amines with one or more additional polar functional groups may be a hydrocarbon, especially an alkane, substituted by the amino group and the additional polar group(s). The amino group may be substituted or unsubstituted and, excluding amino substituents, the polar base may contain, for example, up to 10 carbon atoms; usually there are no less than three such carbon atoms, e.g. 4, 5 or 6. Aminosugars are included in this category of polar bases. Basic amino acids, e.g. lysine or arginine, are also included in this category.

The disclosure includes products comprising salts of the formula (X) 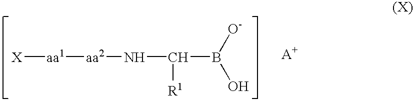
where aa¹, aa², X and R¹ are as defined previously and A⁺ is the protonated form of a pharmaceutically acceptable amine, as well as salts in which both hydroxy groups of the boronate group are in salt form (preferably with another identical A⁺ group) and mixtures of such salts. In one class of such products, A⁺ is the protonated form of an amine described in section 2B(i) below; in another class A⁺ is the protonated form of an amine described in 2B(ii) below. Also included are products in which R¹ is replaced by another R⁹ group.

Two illustrative classes of amine base are described in sections 4B(i) and 4B(ii) below. Particularly preferred are acid salts (in which one of the two boronic —OH groups is deprotonated). Most commonly, the salts contain a single type of amine counter-ion (disregarding trace contaminants) but the disclosure contemplates salts containing mixtures of amine counter-ions; in one sub-class, the different counter-ions all fall within the sub-section 4B(i) family described below or, as the case may be, in the sub-section 4B(ii) family below; in another subclass, the salts comprise a mixture of organic counter-ions which are not all from the same family (4B(i) or 4B(ii)).

4B(i) Aminosugars
The identity of the aminosugar is not critical. Preferred aminosugars include ring-opened sugars, especially glucamines. Cyclic aminosugars are also envisaged as useful. One class of the aminosugars is N-unsubstituted and another, preferred, class is N-substituted by one or two N-substituents (e.g. one). Suitable substituents are hydrocarbyl groups, for example and without limitation containing from 1 to 12 carbon atoms; the substituents may comprise alkyl or aryl moieties or both. Exemplary substituents are C₁, C₂, C₃, C₄, C₅, C₆, C₇ and C₈ alkyl groups, in particular methyl and ethyl, of which methyl is illustrative. Data indicate that aminosugars, especially N-methyl-D-glucamine, are of surprisingly high solubility.
A most preferred aminosugar is N-methyl-D-glucamine:

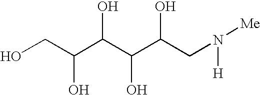

4B(ii) Other Amines
Other suitable amines include amino acids (whether naturally occurring or not) whose side chain is substituted by an amino group, especially lysine.
Some amines are compounds of formula (XI): 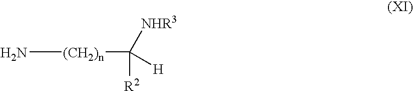
where n, R² and R³ are as defined in relation to formula (IV). The compounds of formula (VI) are usually of L-configuration. The compounds of formula (VI) are lysine (n=4; R²=carboxyl; R³=H) and lysine derivatives or analogues. A most preferred amine is L-lysine.
Other suitable amines are nitrogen-containing heterocycles. At least usually, such heterocyclic compounds are alicyclic; one class of the heterocyclic compounds is N-substituted and another, preferred, class is N-unsubstituted. The heterocycles may contain 6 ring-forming atoms, as in the cases of piperidine, piperazine and morpholine. One class of amines includes N-containing heterocycles substituted by polar substituents, especially hydroxy, e.g. 1, 2 or 3 times.
The disclosure therefore includes amines other than aminosugars which have one or more (e.g. 1, 2, 3, 4, 5 or 6) polar substituents, especially hydroxy, in addition to one amine group. Such compounds may have a ratio of (amino plus hydroxy groups):carbon atoms of 1:2 to 1:1, the latter ratio being particularly preferred.

Also to be mentioned as well as base addition salts are acid addition salts. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate.

The disclosure includes mixed salts, i.e. salts containing a mixture of boropeptide moieties and/or counterions but single salts are preferred.

The salts in solid form may contain a solvent, e.g. water. There are included a class of products in which the salts are essentially anhydrous. Also included is a class in which the salts are hydrates.

Synthetic Methods

1. Peptide/Peptidomimetic Synthesis

The synthesis of boropeptides, including, for example, Cbz-D-Phe-Pro-BoroMpg-OPinacol is familiar to those skilled in the art and described in the prior art mentioned above, including Claeson et al (U.S. Pat. No. 5,574,014 and others) and Kakkar et al (WO 92/07869 and family members including U.S. Pat. No. 5,648,338). It is described also by Elgendy et al Adv. Exp. Med. Biol. (USA) 340:173-178, 1993; Claeson, G. et al Biochem. J. 290:309-312, 1993; Deadman et al J. Enzyme Inhibition 9:29-41, 1995, and by Deadman et al J. Med. Chem. 38:1511-1522, 1995.

By way of example, a P2 amino acid, e.g. proline, is coupled with a compound which will form the P3 residue, e.g. Cbz-4-fluorophenylalanine (commercially available), under peptide coupling conditions, to form a dipeptide.

The end product dipeptide is reacted with an aminoboronate corresponding to the desired P1 residue, e.g. boroMpg, to form the final product

Stereoselective synthesis with S or R configuration at the chiral B-terminal carbon may be conducted using established methodology (Elgendy et al Tetrahedron. Lett. 33:4209-4212, 1992; WO 92/07869 and family members including U.S. Pat. No. 5,648,338) using (+) or (−)-pinanediol as the chiral director (Matteson et al J. Am. Chem. Soc. 108:810-819, 1986; Matteson et al Organometallics. 3:1284-1288, 1984). Another approach is to resolve the requisite aminoboronate intermediate (e.g. Mpg-BOPinacol) to selectively obtain the desired (R)-isomer and couple it to the dipeptide moiety (e.g. Cbz-(R)-4-F-Phe-(S)-Pro) which will form the remainder of the molecule.

For additional information on peptide synthesis, the skilled reader is referred to “The Peptides”, Vol. 1-3, Edited by Erhard Gross, Johannes Meienhofer, 1979 (Vol. 1), 1980 (Vol. 2), 1981 (Vol. 3), Academic Press.

The boropeptides may be synthesised initially in the form of boronic acid esters, particularly esters with diols. Such diol esters may be converted to the peptide boronic acid as described next.

2. Ester to Acid Conversion

A peptide boronate ester such as Cbz-(R)-4-F-Phe-Pro-boroMpg-OPinacol may be hydrolysed to form the corresponding acid.

A novel technique for converting a diol ester of a peptide boronic acid of formula (I) into the acid comprises dissolving the diol ester in an ether and particularly a dialkyl ether, reacting the thus-dissolved diol with a diolamine, for example a dialkanolamine, to form a product precipitate, recovering the precipitate, dissolving it in a polar organic solvent and reacting the thus-dissolved product with an aqueous medium, e.g. an aqueous acid, to form the peptide boronic acid. The boronic acid may be recovered from the organic layer of the mixture resulting from the reaction, for example by removing the solvent, e.g. by evaporation under vacuum or distillation. The reaction between the diol ester and the diolamine may be carried out under reflux, for example.

The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexylethanediol.

The alkyl groups of the dialkyl ether preferably have 1, 2, 3 or 4 carbon atoms and the alkyl groups may be the same or different. An exemplary ether is diethyl ether.

The alkyl groups of the dialkanolamine preferably have 1, 2, 3 or 4 carbon atoms and the alkyl groups may be the same or different. An exemplary dialkanolamine is diethanolamine. The diethanolamine/boronic acid reaction product hydrolyses in water at room temperature and the rate of hydrolysis may be accelerated by adding acid or base.

The polar organic solvent is preferably CHCl₃. Other examples are polyhalogenated alkanes generally and ethyl acetate. In principle, any polar organic solvent is acceptable other than alcohols.

The aqueous acid is suitably a strong inorganic acid at a pH in the region of 1 such as hydrochloric acid, for example.

After reaction with the acid, the reaction mixture is suitably washed with, for example, NH₄Cl or another mild base.

An example of a specific procedure is as follows

1. The pinacol or pinanediol ester of the selected peptide boronic acid is dissolved in diethylether.

2. Diethanolamine is added and the mixture is refluxed at 40° C.

3. The precipitated product is removed (filtered), washed (usually several times) with diethyl ether or another polar organic solvent other than an alcohol, and dried (e.g. by evaporation under vacuum).

4. The dry product is dissolved in a polar organic solvent other than an alcohol, e.g. CHCl₃. Aqueous acid or base is added, e.g. hydrochloric acid (pH 1), and the mixture is stirred for e.g. approximately 1 h at room temperature.

5. The organic layer is removed and washed with NH₄Cl solution.

6. The organic solvent is distilled off and the residual solid product is dried.

The above process results in the formation of what may conveniently be referred to as a “diolamine adduct” of the peptide boronic acids of formula (I), especially such adducts with diethanolamine, and such adducts are themselves included in the disclosure. The molecular structure of such adducts is not known: they might comprise a compound in which the two oxygens and the nitrogen of the diolamine are all coordinated to the boron; they might comprise ions. The adducts are however considered to be esters. A particular novel product included in the disclosure is that obtainable by reacting a pinacol or pinanediol ester of a compound of Formula VIII, particularly Cbz-(R)-4-F-Phe-(S)-Pro-(R)-boroMpg.

It will be appreciated that the aforegoing technique comprises an example of a method for recovering an organoboronic acid product, the method comprising providing in a solvent a dissolved mixture comprising the organoboronic acid in a soluble form and a compound having two hydroxy groups and an amino group (i.e. a diolamine), causing or allowing the organoboronic acid and the diolamine to react to form a precipitate, and recovering the precipitate. The soluble form of the organoboronic acid may be a diol ester, as discussed above. The solvent may be an ether, as discussed above. The organoboronic acid may be one of the organoboronic acids referred to in this specification, for example it may be of Formula (I) or (III). The method described in this paragraph is novel and forms an aspect of the disclosure. A recovery method is filtration.

The reaction between the diolamine and the soluble form of the organoboronic acid is suitable carried out at an elevated temperature, for example under reflux.

Another aspect of the disclosure is a method for recovering an organoboron species, comprising
providing, in a form soluble in an ether, an organoboronic acid, for example a drug such as, e.g., a compound of formula (III);
forming a solution of the soluble form in the ether;
combining the solution with a dialkanolamine and allowing or causing the dialkanolamine to react with the soluble form of the organoboronic acid to form an insoluble precipitate; and
recovering the precipitate.

The term “soluble” in the preceding paragraph refers to species which are substantially more soluble in the reaction medium than is the precipitated product. In variants of the method, the ether is replaced by toluene or another aromatic solvent.

The diethanolamine precipitation technique described above is an example of another novel method, which is a method for recovering from ether solution a pinacol or pinanediol ester of a peptide boronic acid, comprising dissolving diethanolamine in the solution, allowing or causing a precipitate to form and recovering the precipitate. The disclosure encompasses variants of this methods in which another diol than pinacol or pinanediol is used.

The precipitated material, i.e. the “adduct”, may be converted into the free organoboronic acid, for example by contacting it with an acid. The acid may be an aqueous acid, for example an aqueous inorganic acid, e.g. as described above. The precipitate may be dissolved, for example in an organic solvent, prior to being contacted with the acid.

The disclosure therefore provides a method for making an organoboronic acid, comprising converting its diolamine reaction product to the acid.

The acid resulting from the methods described in the previous two paragraphs may be converted to a salt of the acid with a multivalent metal, which salt may in turn be formulated into a pharmaceutical composition in parenteral dosage form.

3. Salt Synthesis

Acid addition salts may be prepared by contacting the boronic acid compound with a pharmaceutically acceptable acid, notably a strong acid.

In general, the base addition salts may be prepared by contacting the relevant peptide boronic acid with a strong base appropriate to form the desired salt. In the case of metal salts, the metal hydroxides are suitable bases (alternatively, metal carbonates might be used, for example), whilst sometimes it is more convenient to contact the acid with a relevant metal alkoxide (e.g. methoxide), for which purpose the corresponding alkanol is a suitable solvent. Salts with organic bases may be prepared by contacting the peptide boronic acid with the organic base itself. Illustrative salts are acid salts (one —BOH proton replaced) and, to make acid salts with a monovalent cation, the acid and the base are suitably reacted in substantially equimolar quantities. Generally stated, therefore, the usual acid:base molar ratio is substantially n:1, where n is the valency of the cation of the base.

In one procedure, a solution of the peptide boronic acid in a water-miscible organic solvent, for example acetonitrile or an alcohol (e.g. ethanol, methanol, a propanol, for example iso-propanol, or another alkanol), is combined with an aqueous solution of the base. The acid and the base are allowed to react and the salt is recovered. The reaction is typically carried out at ambient temperature (e.g. at a temperature of from 15 to 30° C., e.g. 15 to 25° C.), but an elevated temperature may be used, for example up to the boiling point of the reaction mixture but more usually lower, e.g. a temperature of up to 40° C. or 50° C. The reaction mixture may be allowed to stand or be agitated (usually stirred).

The time during which the acid and the base are allowed to react is not critical but it has been found desirable to maintain the reaction mixture for at least one hour. A period of from one to two hours is usually suitable but longer reaction times may be employed.

The salt may be recovered from the reaction mixture by any suitable method, for example evaporation or precipitation. Precipitation may be carried out by adding an excess of a miscible solvent in which the salt has limited solubility. In one preferred technique, the salt is recovered by evacuating the reaction mixture to dryness. The salt is preferably thereafter purified, for example by redissolving the salt before filtering the resulting solution and drying it, for example by evacuating it to dryness. The redissolution may be performed using water, e.g. distilled water. The salt may then be further purified, for example in order to remove residual water by further redissolution in a suitable solvent, which is advantageously ethyl acetate or THF followed by evaporating to dryness. The purification procedure may be carried out at ambient temperature (say, 15 to 30° C., e.g. 15 to 25° C.), or at a modestly elevated temperature, such as e.g. a temperature not exceeding 40° C. or 50° C.; for example the salt may be dissolved in water and/or solvent by agitating with or without warming to, for example, 37° C.

Also included is a method for drying the salts of the disclosure and other peptide boronic acid salts, comprising dissolving them in an organic solvent, e.g. ethyl acetate or THF, and then evaporating to dryness, e.g. by evacuation.

Generally, preferred solvents for use in purifying the salts are ethyl acetate or THF, or perhaps another organic solvent.

A general procedure for synthesising salts of Cbz-4-F-Phe-Pro-BoroMpg-OH is as follows:

Cbz-4-F-Phe-Pro-BoroMpg-OH (38.1 mM) is dissolved in acetonitrile (200 ml) with stirring at room temperature. To this solution is added the requisite base in solution in distilled water (190 ml); the base is added as a 0.2M solution for a monovalent cation. The resultant clear solution is allowed to react for example by being left to stand or being agitated, for a usual period, in either case, of from one to two hours. The reaction is typically carried out at ambient temperature (e.g. 15-30° C., e.g. 15 to 25° C.) but alternatively the temperature may be elevated (e.g. up to 30° C., 40° C. or 50° C.). The reaction mixture is then evacuated to dryness under vacuum with its temperature not exceeding 37° C. The product is redissolved in the minimum amount of distilled water necessary (200 ml to 4 L), typically with warming (e.g. to 30-40° C.), usually for up to 2 hours. The solution is filtered, suitably through filter paper, and evacuated to dryness, again with the temperature of the solution not exceeding 37° C., or freeze dried. The resultant product is dried under vacuum overnight to normally yield a solid. If the product is present as an oil or tacky solid then it is dissolved in ethyl acetate and evacuated to dryness to produce the product as a solid.

In variations of the aforegoing general procedure, the acetonitrile is replaced by another water-miscible organic solvent, notably an alcohol, as discussed above, especially ethanol, methanol, iso-propanol or another propanol.

Where a boronic acid salt is less soluble in a selected reaction medium for salt formation such that its direct preparation from the corresponding acid and base is inconvenient, the less soluble salt may be prepared from a salt more soluble in the reaction medium.

There is provided also the use of a boronic acid to make a salt of the disclosure. Included also is a method of preparing a product of the disclosure, comprising contacting a boronic acid, e.g. of formula (I), (II) or (III), with a base capable of making such a salt.

The peptide boronic acid of formula (I) used to prepare the pharmaceutical preparations is typically of GLP or GMP quality, or in compliance with GLP (good laboratory practice) or GMP (good manufacturing practice); such acids are included in the disclosure.

Similarly the acids are usually sterile and/or acceptable for pharmaceutical use, and one aspect of the disclosure reside in a composition of matter which is sterile or acceptable for pharmaceutical use, or both, and comprises a peptide boronic acid of formula (I). Such a composition of matter may be in particulate form or in the form of a liquid solution or dispersion.

The acid may be in isolated form and such isolated acids are included in the disclosure.

4. Separation of Stereoisomers

The stereoisomers of a peptide boronic ester or a synthetic intermediate aminoboronate may be resolved in, for example, any known way. In particular, stereoisomers of boronic esters may be resolved by HPLC.

5. Specific Synthesis

This method addresses the problems of controlling C—B bond cleavage in organoboronic compounds as well as providing chirally purified on a commercial scale. In this regard, it has been found that C—B bonds seem to be cleaved by a non-oxidative mechanism which occurs in the presence of many solvents, including water and e.g. aqueous acids and bases, amongst others.

Chirally-selective precipitation may be used to recover organoboronic acids in high purity.

Thus C—B bond cleavage may be controlled by:
Selection of acetonitrile as a solvent, where a solvent is required in processing and acetonitrile has the necessary solvation power; in particular acetonitrile is selected in process where a polar solvent is desirable or necessary.
Avoiding excessive contact with water.

In terms of tripeptide boronate salt production, therefore, the disclosure includes processes comprising one, two or three of the following features:
(i) resolution of the (R,S,S) and (R,S,R) epimers of the borotripeptide by chirally selective precipitation using diethanolamine and conveniently, but not necessarily, using as starting material TRI 50f in the form of an ester, for example the pinacol ester;
(ii) control of the duration and/or conditions of hydrolysis of the borotripeptide diethanolamine ester, for example as obtained by such precipitation, to control C—B bond breakage;
(iii) use of acetonitrile as solvent for the borotripeptide, for example as obtained by such hydrolysis, for the purposes of reacting the borotripeptide with a base to form the salt. Another favourable solvent can be tetrahydrofuran.

As an optional, or even stand-alone, fourth feature, the borotripeptide base addition salts may be dried by azeodrying using acetonitrile.

It is considered that C—B bond cleavage may occur by a nucleophilic mechanism, and the disclosure therefore includes methods in which opportunities for nucleophilic attack are minimised.

The above four features, or any one, two or three of them, may be applied to the manufacture and processing of other boronic compounds, particularly acids of formula (I) and their derivatives (e.g. esters and salts).

The disclosure provides in one aspect, therefore, the use of diethanolamine to resolve by selective precipitation the diastereomers of the disclosed boronic acids, exemplified below by reference to boronic acids of formula (I). The starting material may be an acid (I) or a derivative thereof capable of forming a diethanolamine ester of the boronic acid. The precipitation selects acids having a chiral centre C* of (R) configuration as precipitate. The precipitate may be recovered and converted to the corresponding boronic acid or a salt thereof. The salt may be made into a pharmaceutical formulation.

For optimised chiral purity and yield, the diethanolamine may be used in an amount of about 1.25±0.1 equivalents based on initial equivalents of boronic acid having a chiral centre C* of (R) configuration.

The initial boronic acid or acid derivative may for example comprise from 50% to 60% molecules having chiral centre C* of (R)-configuration and from 40% to 50% molecules having chiral centre C* of (S)-configuration.

The method opens the way to commercialisation of the boronic acids (I) and their derivatives, particularly salts, as pharmaceuticals. Commercial scale products and activities using the boronic acids (I) and their derivatives are therefore provided.

In one embodiment, there is provided a process for separating diastereomers of a boronic acid of formula (I), comprising:
combining in diethylether solution (A) a boronic species selected from the boronic acid (I) and its esters, the boronic species including molecules having a chiral centre C* of (R) configuration and molecules having a chiral centre C* of (S) configuration, and (B) diethanolamine, the diethanolamine being in an amount of about 1.25±0.1 equivalents based on the boronic species in which the chiral centre C* is of (R) configuration, and mixing to form a mixture;
causing or allowing the boronic species and the diethanolamine to react until a precipitate forms; and
recovering the precipitate.

When the starting material is an ester, it may be an ester of the boronic acid with an alcohol selected from the group consisting of alcohols whose sole potential electron donor heteroatoms are oxygens which, in the boronic ester, correspond to the oxygens of the ester functional group.

In some methods, the diethanolamine is in an amount of from 1.2 to 1.3 equivalents based on the boronic species in which chiral centre C* is of (R) configuration.

There are included processes in which the boronate species is an ester of the boronic acid and a diol, in particular a diol which is not sterically hindered. As exemplary diols may be mentioned pinacol, neopentylglycol, 1,2-ethanediol, 1,2-propanediol, 1,3-propanediol, 2,3-butanediol, 1,2-diisopropylethanediol, or 5,6-decanediol. A particular diol is pinacol.

The boronic species and the diethanolamine may be caused to react by heating the mixture to an elevated temperature, for example the mixture may be refluxed. e.g. for at least 10 hours.

The precipitate may be recovered by filtration. The recovered precipitate may be washed with diethylether. The recovered precipitate, after washing if such takes places, may be dissolved in a solvent selected from CH₂Cl₂ and CHCl₃ and reprecipitated by combining the resulting solution with diethylether. A particular solvent is CH₂Cl₂.

The recovered precipitate may be converted to the acid of formula (I), suitably by hydrolysis, for example by dissolving the precipitate in an organic solvent selected from e.g. halohydrocarbons and combinations thereof, agitating the resulting solution with an aqueous liquid, e.g. an aqueous acid having a pH of below 3, whereby the dissolved precipitate is converted to the formula (I) acid, and recovering the formula (I) acid by evaporation. The organic solvent may be CH₂Cl₂ or CHCl₃. A particular solvent is CH₂Cl₂. In some processes, organic solvent is further evaporated from the recovered formula (I) acid.

The disclosure includes methods in which an ester of a boronic acid (I), particularly a diethanolamine ester, is hydrolysed in a manner which controls C—B bond cleavage. In particular, this involves limiting the period of hydrolysis at the selected temperature. In the case of diethanolamine ester hydrolysis, the hydrolysis is suitably carried out at room temperature, or less, for a period not exceeding about 30 minutes, e.g. not exceeding about 20 minutes, and optimally of about 20 minutes.

Thus the recovered precipitate referred to in the last paragraph but one may be hydrolysed using an aqueous acid, particularly 2% hydrochloric acid or another mineral acid of similar pH, for no more than about 30 minutes at about room temperature, or less. Suitably, the precipitate is dissolved in a non-nucleophilic organic solvent (e.g. a halohydrocarbon or halohydrocarbon mixture for example CH₂Cl₂) and the resulting solution is contacted with the aqueous acid for a period as previously described. The precipitate is thereby hydrolysed to form the free acid of formula (I), which remains in the organic solvent. The organic solvent may be separated from the aqueous medium and then evaporated to obtain solid acid of formula I.

There are included processes in which a formula (I) acid, for example obtained as described in the preceding paragraph, is dried. In a class of processes, the formula (I) acid is dried when it is in the organic solvent by contacting the solvent with a hygroscopic solid.

Included are processes in which the formula (I) acid, when in the organic solvent, is washed with an aqueous ammonium salt.

Chirally purified boronic acid may be converted to a pharmaceutically acceptable base addition salt thereof, in particular by dissolving the acid in acetonitrile, combining the resultant solution with an aqueous solution or suspension of a pharmaceutically acceptable base, and causing or allowing the base and the acid to react, then evaporating to dryness to obtain an evaporation residue. The step of causing or allowing the acid and the base to react may comprise agitating the combination of the acetonitrile solution of the acid and the aqueous solution or suspension of the base at a temperature of not more than 35° C. and often of not more than 30° C., e.g. not more than 25° C.; an optimal temperature is room temperature, in which case a reaction time of about 2 hours might be appropriate. The process may further comprise:
(i) redissolving the evaporation residue in acetonitrile and evaporating the resulting solution to dryness; and
(ii) repeating step (i) as often as necessary to obtain a dry evaporation residue.

In some processes the dry evaporation residue is dissolved in acetonitrile or tetrahydrofuran to form a solution, and the solution is combined with (e.g. slowly added to, at a rate sufficiently slow to avoid lump formation) a 3:1 to 1:3 v/v mixture of diethylether and an aliphatic or cycloaliphatic solvent to form a precipitate, said solution being added to the diethylether/(cyclo)aliphatic solvent mixture in a ratio (solution:mixture) of from 1:5 to 1:15 v/v. The precipitate is recovered and some or substantially all remaining solvent is removed from the recovered precipitate whilst maintaining the temperature at no more than 35° C., e.g. is removed under reduced pressure. Included are processes in which the temperature at the start of the drying process is about 10° C. and is increased during the process to 35° C. The aliphatic or cycloaliphatic solvent may have 6, 7 or 8 carbon atoms; the solvent may be an alkane, for example an n-alkane, e.g. n-heptane. Some reactions may be carried out at ambient temperature, which may e.g. be 15-30° C., e.g. 20-30° C.; sometimes ambient temperature may be room temperature.

The salts produced by the invention may contain a trace amount of the aliphatic or cycloaliphatic solvent, e.g. an amount of less than 0.1%, particularly less than 0.01%, for example an amount of about 0.005%.

In the process for making the salt, the base may comprise a cation of valency n and be used in a stoichiometry (boronic acid:base) of about n:1. In particular processes, the base is an alkali metal or alkaline earth metal base, for example an alkali metal hydroxide or an alkaline earth metal hydroxide. As one base may be mentioned sodium hydroxide. As another base may be mentioned calcium hydroxide. The disclosure includes processes in which the base is sodium hydroxide and the dry evaporation residue is dissolved in acetonitrile. The disclosure includes processes in which the base is calcium hydroxide and the dry evaporation residue is dissolved in tetrahydrofuran.

The disclosure is not limited as to the method by which the boronic acids of Formula (I) are obtained (for example as an ester thereof). However, in one class of subject matter, the Formula (I) acid has an R¹ group of the formula —(CH₂)ₛ—O—R³ in which R³ is methyl or ethyl and s is independently 2, 3 or 4, and the Formula (I) acid is prepared via an intermediate of Formula (XXV):
(HO)₂B—(CH₂)ₛ—O—R³ (XXV),
which intermediate is made by reaction between a borate ester and a suitable 1-metalloalkoxyalkane.

A novel aspect of the disclosure comprises the Formula (XXV) intermediates.

The Formula (XXV) intermediates may be made by reacting a 1-metalloalkoxyalkane, where the alkoxyalkane is of the formula —(CH₂)ₛ—O—R³, with a borate ester to form a compound of Formula (XXV).

It will be appreciated that the above method provides a general procedure for making alkoxyalkylboronic acids, which may be presented by the formula R^{Z}—O—R^{Y}—B(OH)₂. Such alkoxyalkylboronic acids may be converted to aminoboronates, and the aminoboronates may be derivatised at their amino group to form an amide bond linked to another moiety. In other words, the aminoboronates may be converted to boropeptides. The method will now be described further with non-limiting reference to compounds of Formula (XXV).

The starting materials for the reaction may be a metalloalkoxyalkane, e.g. a Grignard reagent, obtainable from 1-haloalkoxyalkane of the formula Hal-(CH₂)ₛ—O—R³ (where Hal is a halogen) and a borate ester. The metal is in particular magnesium. Another metal is lithium, in which case the metallo reagent may be prepared by reacting the 1-haloalkoxyalkane with butyl lithium. Where the method includes preparation of the metallo reagent from the haloalkoxyalkane, the haloalkoxyalkane may be a chloroalkoxyalkane; the corresponding bromo compounds may also be used. To make a Grignard reagent, magnesium may be reacted with the haloalkoxyalkane.

Suitable borate esters are esters of mono- and di-functional alcohols (e.g. of EtOH, MeOH, BuOH, pinacol, glycol, pinanediol etc). For example, the ester may be of the formula B(OR^{a})(OR^{b})(OR^{c}) where R^{a}, R^{b} and R^{c} and C₁-C₄ alkyl and may be the same as each other.

An exemplary procedure for making a Formula (XXV) intermediate, illustrated with reference to methoxypropane as the alkoxyalkane species, is:

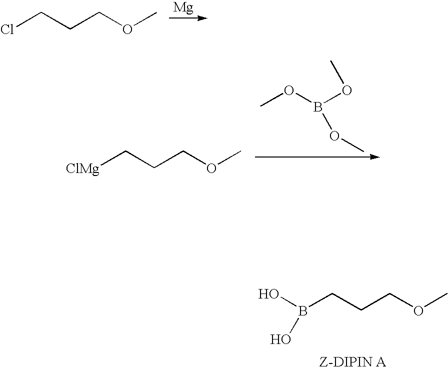

The reactions are suitably carried out in an organic solvent, e.g. THF.

The above-described procedure for making alkoxyalkylboronic acids avoids generation of Impurity IV (see above), or its analogues in those cases where the end product is not TRI 50f or a derivative (salt, ester etc) thereof. The procedure therefore provides a unique route to making TRI 50f, its esters and salts, uncontaminated by Impurity IV, and for making other aminoboronic acids which are substituted α- to the boron by an alkoxyalkyl group and are uncontaminated by impurities analogous to Impurity IV.

An alkoxyalkylboronic acid, i.e. a compound which may be represented by the formula R^{Z}—O—R^{Y}—B(OH)₂, may be converted to an aminoboronic compound, for example a boropeptide, by any suitable procedure, e.g. one known in the art. A reaction scheme for making alkoxyalkylboronic acids into aminoboronates, and for converting aminoboronates into peptide boronates is illustrated with reference to synthesis of TRI 50f at the start of the Examples of this specification. The reaction scheme may be modified as desired, e.g.: diethanolamine precipitation and subsequent steps may be omitted, and/or reagent substitutions may be made. For example, pinacol may be replaced by another diol. LDA is a non-nucleophilic strong base and may be replaced by another such base. Other examples include, but are not limited to, lithium diisopropylamide, lithium 2,2,6,6-tetramethylpiperidine, 1-lithium 4-methylpiperazide, 1,4-dilithium piperazide, lithium bis(trimethyl-silyl)amide, sodium bis(trimethylsilyl)amide, potassium bis(trimethylsilyl)amide, isopropyl magnesium chloride, phenyl magnesium chloride, lithium diethylamide, and potassium tert-butoxide. The reactions may be carried out in any suitable solvent: where n-heptane is used in the Examples, it may be replaced by another inert non-polar solvent, e.g. another aliphatic or cycloaliphatic solvent, for example an alkane, e.g. an n-alkane.

The product is an aminoboronate of Formula (XXI) 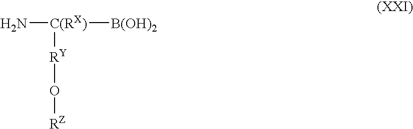
wherein
R^{X} is H or a substituent which does not prevent synthesis;
R^{Y} is alkylene; and
R^{Z} is alkyl,
the process comprising reacting a 1-metalloalkoxyalkane with a borate ester to form a boronic acid of the formula R^{Z}—O—R^{Y}—B(OH)₂, esterifying the acid, contacting the esterified acid with CH₂Cl₂ and ZnCl₂ in the presence of a strong base, contacting the resultant produce with LIHMDS and in turn contacting the resultant product with hydrogen chloride.

The product is free of contaminant of Formula (XXII):
H₂N—C(R^{X})(R^{Y})—B(OH)₂ (XXII).

The aminoboronate (XXI) may be reacted with an amino acid or peptide (which in either case may be suitably protected) to form a peptide boronate. In general terms, therefore, the disclosure includes peptidoboronic acids of Formula (XXIII): 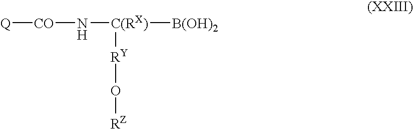
Q-CO comprises at least an amino acid residue;
R^{X} is H or a substituent which does not prevent synthesis;
R^{Y} is alkylene;
R^{Z} is alkyl,
which organoboronic acid is free of an impurity of Formula (XXIV):

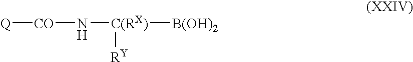

The disclosure further includes derivatives of Formula (XXIII) acids (e.g. acid or base addition salts, esters) which are free of Formula (XXIV) impurity and derivatives thereof.

The exact identity of R^{Y} and R^{Z} is dependent on the identity of the end product, and not part of the process or its benefits.

It will be appreciated from the aforegoing that the above described methods may be used in the manufacture of organoboronic acids salts as described. It is not necessary for sequential steps to be carried out as one operation or at the same site: they may be performed in this way or different processes (different parts of the overall synthesis) may be distributed in time and/or space. Particular end product salts are monosodium, monolithium, hemicalcium and hemimagnesium salts, for example of TRI 50S.

Generally, the reactions may suitably be carried out with a non-nucleophilic solvent. Where a nucleophilic solvent is present, minimum contact is preferred, for example in the case of hydrolysis of diethanolamine esters.

6. Products of Specific Synthesis 5

The products of the invention include inter alia boronic acids, diethanolamine esters and salts obtainable by (having the characteristics of a product obtained by) the disclosed methods. Also included are products obtained directly or indirectly by the disclosed methods.

Particular products of the invention are base addition salts of a boronic acid of formula (I) having the chiral purity of such salt when prepared by a method described herein. Other products are base addition salts of a boronic acid of formula (I) having the purity of such salt when prepared by a method described herein.

Product identities will be apparent from the preceding description and the following examples. In addition, products of the disclosure are described in the claims. Of particular note are the data in Example 9, indicating that the processes of the invention can remarkably achieve end product salts free of impurities detectable by HPLC. In other instances, the salts are substantially free of impurities, e.g. at least 98% pure, more usually at least 99% pure, e.g. at least 99.5% pure, in terms of reverse phase (RP) HPLC percentage peak area. Salts may at least 99.3%, 99.4%, 99.5% 99.6%, 99.7%, 99.8% or 99.9% pure, in terms of reverse phase (RP)HPLC percentage peak area. Suitable RP HPLC procedures comply with reference 1 and/or reference 2 and/or reference 3 of Example 43. Included also are products at least substantially free of Impurity I and analogues, products free of Impurity IV and analogues, and products containing small traces of non-polar solvent, e.g. n-heptane. The trace amount of non-polar solvent may be less than 0.2%, 0.1%, 0.05%, 0.01% or 0.005% as determined by GC-headspace chromatography.

Included also are salts containing less than 410 ppm acetonitrile.

Some salts contain impurities of less than 10,000 ppm, 5000 ppm, 1000 ppm, or 500 ppm.

Use of the Products of the Disclosure

The compounds of the disclosure are thrombin inhibitors. They are therefore useful for inhibiting thrombin. There are therefore provided compounds which have potential for controlling haemostasis and especially for inhibiting coagulation, for example in the treatment or prevention of secondary events after myocardial infarction. The medical use of the compounds may be prophylactic (including to treat thrombosis as well as to prevent occurrence of thrombosis) as well as therapeutic (including to prevent re-occurrence of thrombosis or secondary thrombotic events).

The compounds may be employed when an anti-thrombogenic agent is needed. Further, it has been found that the compounds, including those of boronic acids of Formula (III), are beneficial in that the class is useful for treating arterial thrombosis by therapy or prophylaxis. The disclosed compounds are thus indicated in the treatment or prophylaxis of thrombosis and hypercoagulability in blood and tissues of animals including man. The term “thrombosis” includes inter alia atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

It is known that hypercoagulability may lead to thromboembolic diseases.

Examples of venous thromboembolism which may be treated or prevented with compounds of the disclosure include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, compounds of the disclosure are useful for maintaining patency of indwelling catheters.

Examples of cardiogenic thromboembolism which may be treated or prevented with compounds of the disclosure include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

Examples of conditions involving arterial thrombosis include unstable angina (severe constrictive pain in chest of coronary origin), myocardial infarction (heart muscle cell death resulting from insufficient blood supply), ischemic heart disease (local ischemia due to obstruction (such as by arterial narrowing) of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterio-venous (mixed) thrombosis, anti-thrombotic compounds of the disclosure are useful for maintaining patency in arteriovenous shunts.

Other conditions associated with hypercoagulability and thromboembolic diseases which may be mentioned inherited or acquired deficiencies in heparin cofactor II, circulating antiphospholipid antibodies (Lupus anticoagulant), homocysteinemia, heparin induced thrombocytopenia and defects in fibrinolysis.

Particular uses which may be mentioned include the therapeutic and/or prophylactic treatment of venous thrombosis and pulmonary embolism. Preferred indications envisaged for the products of the disclosure (notably the compounds of TRI 50f) include:
Prevention of venous thromboembolic events (e.g. deep vein thrombosis and/or pulmonary embolism). Examples include patients undergoing orthopaedic surgery such as total hip replacement, total knee replacement, major hip or knee surgery; patients undergoing general surgery at high risk for thrombosis, such as abdominal or pelvic surgery for cancer; and in patients bedridden for more than 3 days and with acute cardiac failure, acute respiratory failure, infection.
Prevention of thrombosis in the haemodialysis circuit in patients, in patients with end stage renal disease.
Prevention of cardiovascular events (death, myocardial infarction, etc) in patients with end stage renal disease, whether or not requiring haemodialysis sessions.
Prevention of venous thrombo-embolic events in patients receiving chemotherapy through an indwelling catheter.
Prevention of thromboembolic events in patients undergoing lower limb arterial reconstructive procedures (bypass, endarteriectomy, transluminal angioplasty, etc).
Treatment of venous thromboembolic events.
Prevention of cardiovascular events in acute coronary syndromes (e.g. unstable angina, non Q wave myocardial ischaemia/infarction), in combination with another cardiovascular agent, for example aspirin (acetylsalicylic acid; aspirin is a registered trade mark in Germany), thrombolytics (see below for examples), antiplatelet agents (see below for examples).
Treatment of patients with acute myocardial infarction in combination with acetylsalicylic acid, thrombolytics (see below for examples).
Prevention of thrombosis in apheresis generally, including intermittent apheresis, e.g. extracorporeal liver detoxification.
Flight DVT.
Prevention of thrombosis in procedures involving cardiopulmonary bypass (CPB).
Preventing thrombosis during coronary artery bypass graft (CABG) with or without CPB.
Prevention of thrombosis in extracorporeal blood circuits generally.

The thrombin inhibitors of the disclosure are thus indicated both in the therapeutic and/or prophylactic treatment of all the aforesaid disorders.

In one method, the products of the disclosure are used for the treatment of patients by haemodialysis, by providing the product in the dialysis solution, as described in relation to other thrombin inhibitors in WO 00/41715. The disclosure therefore includes dialysing solutions and dialysing concentrates which comprise a product of the disclosure, as well as a method of treatment by dialysis of a patient in need of such treatment, which method comprises the use of a dialysing solution including a low molecular weight thrombin inhibitor. Also included is the use of an anti-thrombotic product of the disclosure for the manufacture of a medicament for the treatment by dialysis of a patient, in which the anti-thrombotic product of the disclosure is provided in the dialysing solution.

In another method, the products of the disclosure are used to combat undesirable cell proliferation, as described in relation to other thrombin inhibitors in WO 01/41796. The undesirable cell proliferation is typically undesirable hyperplastic cell proliferation, for example proliferation of smooth muscle cells, especially vascular smooth muscle cells. The products of the disclosure particularly find application in the treatment of intimal hyperplasia, one component of which is proliferation of smooth muscle cells. Restenosis can be considered to be due to neointimal hyperplasia; accordingly intimal hyperplasia in the context of the disclosure includes restenosis.

The products of the disclosure are also contemplated for the treatment of ischemic disorders. More particularly, they may be used in the treatment (whether therapeutic or prophylactic) of an ischemic disorder in a patient having, or at risk of, non-valvular atrial fibrillation (NVAF) as described in relation to other thrombin inhibitors in WO 02/36157. Ischemic disorders are conditions whose results include a restriction in blood flow to a part of the body. The term will be understood to include thrombosis and hypercoagulability in blood, tissues and/or organs. Particular uses that may be mentioned include the prevention and/or treatment of ischemic heart disease, myocardial infarction, systemic embolic events in e.g. the kidneys or spleen, and more particularly of cerebral ischemia, including cerebral thrombosis, cerebral embolism and/or cerebral ischemia associated with non-cerebral thrombosis or embolism (in other words the treatment (whether therapeutic or prophylactic) of thrombotic or ischemic stroke and of transient ischemic attack), particularly in patients with, or at risk of, NVAF.

The products of the disclosure are also contemplated for the treatment of rheumatic/arthritic disorders, as described in relation to other thrombin inhibitors in WO 03/007984. Thus, the products of the disclosure may be used in the treatment of chronic arthritis, rheumatoid arthritis, osteoarthritis or ankylosing spondylitis

Moreover, the products of the disclosure are expected to have utility in prophylaxis of re-occlusion (i.e. thrombosis) after thrombolysis, percutaneous trans-luminal angioplasty (PTA) and coronary bypass operations; the prevention of re-thrombosis after microsurgery and vascular surgery in general. Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis.

The products of the disclosure are further indicated in the treatment of conditions where there is an undesirable excess of thrombin without signs of hypercoagulability, for example in neurodegenerative diseases such as Alzheimer's disease. In addition to its effects on the coagulation process, thrombin is known to activate a large number of cells (such as neutrophils, fibroblasts, endothelial cells and smooth muscle cells). Therefore, the compounds of the disclosure may also be useful for the therapeutic and/or prophylactic treatment of idiopathic and adult respiratory distress syndrome, pulmonary fibrosis following treatment with radiation or chemotherapy, septic shock, septicaemia, inflammatory responses, which include, but are not limited to, edema, acute or chronic atherosclerosis such as coronary arterial disease, cerebral arterial disease, peripheral arterial disease, reperfusion damage, and restenosis after percutaneous trans-luminal angioplasty (PTA).

The compounds may also be useful in the treatment of pancreatitis.

The compounds described herein are further considered to be useful for inhibiting platelet procoagulant activity. The disclosure provides a method for inhibiting platelet pro-coagulant activity by administering a salt of a boronic acid described herein to a mammal at risk of, or suffering from, arterial thrombosis, particularly a human patient. Also provided is the use of such compounds for the manufacture of medicaments for inhibiting platelet procoagulant activity.

The use of products of the disclosure as inhibitors of platelet pro-coagulant activity is predicated on the observation that the boronic acids described herein are indicated to be effective at inhibiting arterial thrombosis as well as venous thrombosis.

Indications involving arterial thrombosis include acute coronary syndromes (especially myocardial infarction and unstable angina), cerebrovascular thrombosis and peripheral arterial occlusion and arterial thrombosis occurring as a result of atrial fibrillation, valvular heart disease, arterio-venous shunts, indwelling catheters or coronary stents. Accordingly, in another aspect there is provided a method of treating a disease or condition selected from this group of indications, comprising administering to a mammal, especially a human patient, a salt of the disclosure. The disclosure includes products for use in an arterial environment, e.g. a coronary stent or other arterial implant, having a coating which comprises a salt according to the disclosure.

The salts of the disclosure may be used prophylactically to treat an individual believed to be at risk of suffering from arterial thrombosis or a condition or disease involving arterial thrombosis or therapeutically (including to prevent re-occurrence of thrombosis or secondary thrombotic events).

There is therefore included the use of selective thrombin inhibitors (organoboronic acid salts) described herein for treatment of the above disorders by prophylaxis or therapy as well as their use in pharmaceutical formulations and the manufacture of pharmaceutical formulations.

Administration and Pharmaceutical Formulations

1. Aqueous Solutions

It may be desirable to make aqueous solutions of boronic acid drugs for administering them. It has been found possible to form surprisingly concentrated boronate salt solutions (of up to about 600 mg/ml in the case of TRI 50c monosodium salt) at a pH of about 9.5. However, a solution with a pH of 9.5 may be unacceptable or undesirable. Accordingly, a pharmaceutically acceptable organic acid may be included in the particulate formulation in an amount selected to reduce the pH to a value at which the solution is more acceptable but at which a solution of drinkable quantity (e.g. about 50 ml to about 150 ml) may be formed by reconstituting the particulate formulation. As the organic acid may be mentioned citric acid, tartaric acid or malic acid, for example. In many instances, citric acid is chosen.

Experiments have been performed to test the solubility of TRI 50c monosodium salt at different pH values. All the experiments were conducted using a quantity of the salt equivalent to 600 mg TRI 50c free acid. In a first series of experiments, this amount of the salt was dissolved in 50 ml water to form a solution of approximately pH 9.5. Dilute aqueous HCl was added to determine how much the pH could be reduced before precipitation occurred. It was found that the salt tended to precipitate when the pH of the reconstituted solution was reduced below 9 and the pH of a reconstituted liquid having this concentration of salt may therefore be maintained at 9 or more, e.g. 9.2 or more, to keep the salt in solution.

In a second series of experiments, the same amount of the salt was dissolved in 150 ml water, and citric acid was added. It was found that the pH could be reduced to a value of 3.7-3.8 using citric acid before precipitation occurred. In other words if, in the case of a salt dosage equivalent to 600 mg TRI 50c, the patient instructions are to prepare a solution in at least 150 ml water, a quantity of organic acid (e.g. citric acid) can be included in the formulation which will reduce the pH to a value of, say, not less than 4, without a risk of precipitation. Since acid solutions tend to be more palatable than alkaline ones, and citric acid is a common flavouring agent, this behaviour of the salt is highly beneficial. In practical terms, up to 200 mg citric acid may be combined with TRI 50c monosodium salt (600 mg, calculated as TRI 50c) for a preparation to be reconstituted in 150 ml water or more. In general, it is contemplated that the boronate will be formulated to form a reconstituted solution having a pH of from 4 to 8, e.g. 4 to 7, optionally 5 to 6.

Of course, the absolute amount of citric or other acid would be varied with (i) the absolute amount of the salt and (ii) the desired reconstituted volume, in line with the guidance from the above results and such routine experimentation as might be necessary.

The above remarks apply mutatis mutandis to the base addition salts of the disclosed acids.

2. Pharmaceutical Formulations

The disclosed products may be administered to a host, for example, in the case where the drug has anti-thrombogenic activity, to obtain an anti-thrombogenic effect. In the case of larger animals, such as humans, the compounds may be administered alone or in combination with pharmaceutically acceptable diluents, excipients or carriers. The term “pharmaceutically acceptable” includes acceptability for both human and veterinary purposes, of which acceptability for human pharmaceutical use is preferred.

The products of the disclosure may be combined and/or co-administered with any cardiovascular treatment agent. There are large numbers of cardiovascular treatment agents available in commercial use, in clinical evaluation and in pre-clinical development, which could be selected for use with a product of the disclosure for the prevention of cardiovascular-disorders by combination drug therapy. Such agent can be one or more agents selected from, but not limited to several major categories, namely, a lipid-lowering drug, including an IBAT (ileal Na⁺/bile acid cotransporter) inhibitor, a fibrate, niacin, a statin, a CETP (cholesteryl ester transfer protein) inhibitor, and a bile acid sequestrant, an anti-oxidant, including vitamin E and probucol, a IIb/IIIa antagonist (e.g. abciximab, eptifibatide, tirofiban), an aldosterone inhibitor (e.g. spirolactone and epoxymexrenone), an adenosine A2 receptor antagonist (e.g. losartan), an adenosine A3 receptor agonist, a beta-blocker, acetylsalicylic acid, a loop diuretic and an ACE (angiotensin converting enzyme) inhibitor.

The products of the disclosure may be combined and/or co-administered with any antithrombotic agent with a different mechanism of action, such as the antiplatelet agents acetylsalicylic acid, ticlopidine, clopidogrel, thromboxane receptor and/or synthetase inhibitors, prostacyclin mimetics and phosphodiesterase inhibitors and ADP-receptor (P₂ T) antagonists.

The products of the disclosure may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The products of the disclosure may be combined and/or co-administered with a cardioprotectant, for example an adenosine A1 or A3 receptor agonist.

There is also provided a method for treating an inflammatory disease in a patient that comprises treating the patient with a product of the disclosure and an NSAID, e.g., a COX-2 inhibitor. Such diseases include but are not limited to nephritis, systemic lupus, erythematosus, rheumatoid arthritis, glomerulonephritis, vasculitis and sarcoidosis. Accordingly, the anti-thrombotic salts of the disclosure may be combined and/or co-administered with an NSAID.

Typically, therefore, the products described herein may be administered to a host to obtain a thrombin-inhibitory effect, or in any other thrombin-inhibitory or anti-thrombotic context mentioned herein.

Actual dosage levels of active ingredients in the pharmaceutical compositions of this disclosure may be varied so as to obtain an amount of the active compound(s) that is effective to achieve the desired therapeutic response for a particular patient, composition, and mode of administration (referred to herein as a “therapeutically effective amount”). The selected dosage level will depend upon the activity of the particular compound, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

According to a further aspect there is provided a parenteral, especially intravenous, formulation including a product as described herein. The formulation may consist of the salt alone or it may contain additional components, in particular the salt may be in combination with a pharmaceutically acceptable diluent, excipient or carrier, for example a tonicity agent for the purpose of making the formulation substantially isotonic with the body of the subject to receive the formulation, e.g. with human plasma. The formulation may be in ready-to-use form or in a form requiring reconstitution prior to administration. Intravenous formulations may be injected or infused into the patient or, where applicable, into an extracorporeal blood circuit.

It is currently contemplated that, in the case of parenteral administration, for example i.v. administration, of salts of TRI 50c, the salts might for instance be administered in an amount of from 0.5 to 2.5 mg/Kg e.g. over a maximum period of 72 hours, calculated as TRI 50c. Other salts might be administered in equivalent molar amounts. The disclosure is not limited to administration in such quantities or regimens and includes dosages and regimens outside those described in the previous sentence.

In the case of CIHD or other intermittent apheresis, it is contemplated that the disclosed compounds may be administered as i.v. formulations into the extracorporeal blood stream or intravenously, or as oral formulations, e.g. as drinks reconstituted from dry powder sachets or effervescent tablets. The activated clotting time (ACT) is a commonly used parameter for assessing the degree of anticoagulation and the target ACT in CIHD is 150 to 250 sec.

In the case of cardiovascular or cardiac surgery, for example coronary artery bypass grafting (with or without cardiopulmonary bypass) or valve repair or replacement, the patient suffers a large thrombogenic stimulus and higher levels of anticoagulation are required than in apheresis. The amount of anticoagulation can be measured by the activated clotting time (ACT). In experiments conducted using TRI 50c monosodium salt in the dog, it was found that the ACT should be >300 seconds before surgical intervention commences. ACT is a commonly used parameter for assessing the degree of anticoagulation during cardiac surgery. It has been found suitable in settings such as these for administration of the antithrombotic boronic acid salt to commence with a bolus administration of the compound followed by infusion. The rate of administration may be adjusted according to clinical judgement, for example to take account of a patient's weight or other factors, and a rate of administration of 0.1-0.75 mmoles/hour may be mentioned. In the case of dissimilar potencies, say (Ki values outside the range of 5-25 nM), dosage rate may be adjusted accordingly. The disclosure is not limited to administration in such quantities or regimens and includes dosages and regimens outside those described in this paragraph.

It is desirable in CIHD that as little water as possible be added during anticoagulation, since one of the purposes of CIHD is to remove water from the blood. It is therefore contemplated that a relatively soluble antithrombotic will be used, e.g. a sodium salt or a reaction product of a boronic acid and an aminosugar (for example N-methyl-D-glucamine) in the case of CIHD. Other procedures which involve intravenous anticoagulation may be less sensitive to the volume of water injected or infused and less soluble products may be preferred on a balance of factors. Thus, for example, it may in such instances be preferred to administer a salt of a divalent metal such as calcium or zinc for reason of stability. Magnesium is another pharmaceutically acceptable divalent metal. Trivalent metals may also be mentioned.

Examples of the procedures or settings less sensitive to volume of added water referred to in the previous paragraph include:
Surgery, for example cardiovascular or cardiac surgery (e.g. CABG with or without CPB), surgery involving CPB, orthopaedic surgery such as total hip replacement, total knee replacement, major hip or knee surgery; general surgery on patients at high risk of thrombosis, such as abdominal or pelvic surgery for cancer;
Apheresis procedures other than haemodialysis,
Prevention of venous thromboembolic events (e.g. deep vein thrombosis and/or pulmonary embolism). Examples include patients who have suffered or are suspected of having suffered a thrombotic event; and patients bedridden for more than 3 days and with acute cardiac failure, acute respiratory failure, infection
Prevention of venous thrombo-embolic events in patients receiving chemotherapy through an indwelling catheter.
Prevention of thromboembolic events in patients undergoing lower limb arterial reconstructive procedures (bypass, endarteriectomy, transluminal angioplasty, etc).
Treatment of venous thromboembolic events.
Prevention of cardiovascular events in acute coronary syndromes (e.g. unstable angina, non Q wave myocardial ischaemia/infarction), in combination with another cardiovascular agent, for example aspirin (acetylsalicylic acid; aspirin is a registered trade mark in Germany), thrombolytics (see below for examples), antiplatelet agents (see below for examples).
Treatment of patients with acute myocardial infarction in combination with acetylsalicylic acid, thrombolytics (see below for examples)
Other acute treatments used in relation to indications indicated previously, except in the setting of renal failure or disorder.

Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration.

Liquid dosage forms for parenteral administration may include solutions, suspensions, liposome formulations, or emulsions in oily or aqueous vehicles. In addition to the active compounds, the liquid dosage forms may contain other compounds. Tonicity agents (for the purpose of making the formulations substantially isotonic with the subject's body, e.g. with human plasma) such as, for instance, sodium chloride, sodium sulfate, dextrose, mannitol and/or glycerol may be optionally added to the parenteral formulation. A pharmaceutically acceptable buffer may be added to control pH. Thickening or viscosity agents, for instance well known cellulose derivatives (e.g. methylcellulose, carboxymethylcellulose, hydroxyethylcellulose and hydroxypropylmethylcellulose), gelatin and/or acacia, may optionally be added to the parenteral formulation.

Solid dosage forms for parenteral administration may encompass solid and semi-solid forms and may include pellets, powders, granules, patches, and gels. In such solid dosage forms, the active compound is typically mixed with at least one inert, pharmaceutically acceptable excipient or carrier. The disclosed compounds may be presented as solids in finely divided solid form, for example they may be milled or micronised.

The formulations may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The parenteral formulations may be prepared as large volume parenterals (LVPs), e.g. larger than 100 ml, more particularly about 250 ml, of a liquid formulation of the active compound. Examples of LVPs are infusion bags. The parenteral formulations may alternatively be prepared as small volume parenterals (SVPs), e.g. about 100 ml or less of a liquid formulation of the active compound. Examples of SVPs are vials with solution, vials for reconstitution, prefilled syringes for injection and dual chamber syringe devices.

The formulations of the disclosure include those in which the compound is an alkali metal salt, for example a lithium, sodium or potassium salt, of which sodium salts may be mentioned as particular salts. Another class of formulations contains aminosugar salts of the disclosed boronic acids, for example N-methyl-D-glucamine salts. The salts mentioned in this paragraph may be administered as solutions in water, typically containing one or more additives, for example isotonicity agent(s) and/or antioxidant(s). A suitable way to store the salts is in solid form, for example as dry powder, and to make them up into solutions for administration prior to administration.

One class of formulations disclosed herein is intravenous formulations. For intravenously administered formulations, the active compound or compounds can be present at varying concentrations, with a carrier acceptable for parenteral preparations making up the remainder. Particularly, the carrier is water, particularly pyrogen free water, or is aqueous based. Particularly, the carrier for such parenteral preparations is an aqueous solution comprising a tonicity agent, for example a sodium chloride solution.

By “aqueous based” is meant that formulation comprises a solvent which consists of water or of water and water-miscible organic solvent or solvents; as well as containing a compound of disclosure in dissolved form, the solvent may have dissolved therein one or more other substances, for example an antioxidant and/or an isotonicity agent. As organic cosolvents may be mentioned those water-miscible solvents commonly used in the art, for example propyleneglycol, polyethyleneglycol 300, polyethyleneglycol 400 and ethanol. Preferably, organic co-solvents are only used in cases where the active agent is not sufficiently soluble in water for a therapeutically effective amount to be provided in a single dosage form. As previously indicated, the disclosure includes formulations of alkali metal salts of the disclosed boronic acids, e.g. TRI 50f, having a solvent which consists of water.

The solubility of the active compound in the present formulations may be such that the turbidity of the formulation is lower than 50 NTU, e.g. lower than 20 NTU such as lower than 10 NTU.

It is desirable that parenteral formulations are administered at or near physiological pH. It is believed that administration in a formulation at a high pH (i.e., greater than 8) or at a low pH (i.e., less than 5) is undesirable. In particular, it is contemplated that the formulations would be administered at a pH of between 6.0 and 7.0 such as a pH of 6.5.

The parenteral formulation may be purged of air when being packaged. The parenteral formulation may be packaged in a sterile container, e.g. vial, as a solution, suspension, gel, emulsion, solid or a powder. Such formulations may be stored either in ready-to-use form or in a form requiring reconstitution prior to administration.

Parenteral formulations according to the disclosure may be packaged in containers. Containers may be chosen which are made of material which is non-reactive or substantially non-reactive with the parenteral formulation. Glass containers or plastics containers, e.g. plastics infusion bags, may be used. A concern of container systems is the protection they afford a solution against UV degradation. If desired, amber glass employing iron oxide or an opaque cover fitted over the container may afford the appropriate UV protection.

Plastics containers such as plastics infusion bags are advantageous in that they are relatively light weight and non-breakable and thus more easily stored. This is particularly the case for Large Volume parenterals.

The intravenous preparations may be prepared by combining the active compound or compounds with the carrier. After the formulation is mixed, it may be sterilized, for example using known methods. Once the formulation has been sterilized, it is ready to be administered or packaged, particularly in dark packaging (e.g. bottles or plastics packaging), for storage. It is envisaged, however, that the disclosed compounds might not be stored in solution but as dry solids, particularly a finely divided form such as, for example, a lyophilisate, in order to prolong shelf life; this would of course apply to other parenteral formulations, not only intravenous ones.

The intravenous preparations may take the form of large volume parenterals or of small volume parenterals, as described above.

In a specific embodiment, the present disclosure is directed to products, particularly kits, for producing a single-dose administration unit. The products (kits) may each contain both a first container having the active compound (optionally combined with additives, for example anti-oxidant, preservative and, in some instances, tonicity agent) and a second container having the carrier/diluent (for example water, optionally containing one or more additives, for example tonicity agent). As examples of such products may be mentioned single and multi-chambered (e.g. dual-chamber) pre-filled syringes; exemplary pre-filled syringes are available from Vetter GmbH, Ravensburg, Germany. Such dual chamber syringes or binary syringes will have in one chamber a dry preparation including or consisting of the active compound and in another chamber a suitable carrier or diluent such as described herein. The two chambers are joined in such a way that the solid and the liquid mix to form the final solution.

One class of formulations disclosed herein comprises subcutaneous or intradermal formulations (for example formulations for injection) in which the active compound (or active agent combination) is formulated into a parenteral preparation that can be injected subcutaneously or intradermally. The formulation for administration will comprise the active compound and a liquid carrier.

The carrier utilized in a parenteral preparation that will be injected subcutaneously or intradermally may be an aqueous carrier (for example water, typically containing an additive e.g. an antioxidant and/or an isotonicity agent) or a nonaqueous carrier (again one or more additives may be incorporated). As a non-aqueous carrier for such parenteral preparations may be mentioned highly purified olive oil.

The active compound and the carrier are typically combined, for example in a mixer. After the formulation is mixed, it is preferably sterilized, such as with U.V. radiation. Once the formulation has been sterilized, it is ready to be injected or packaged for storage. It is envisaged, however, that the disclosed compounds will not be stored in liquid formulation but as dry solids, in order to prolong shelf life.

For making subcutaneous implants, the active compound may suitably be formulated together with one or more polymers that are gradually eroded or degraded when in use, e.g. silicone polymers, ethylene vinylacetate, polyethylene or polypropylene.

Transdermal formulations may be prepared in the form of matrices or membranes, or as fluid or viscous formulations in oil or hydrogels or as a compressed powder pellet. For transdermal patches, an adhesive which is compatible with the skin may be included, such as polyacrylate, a silicone adhesive or polyisobutylene, as well as a foil made of, e.g., polyethylene, polypropylene, ethylene vinylacetate, polyvinylchloride, polyvinylidene chloride or polyester, and a removable protective foil made from, e.g., polyester or paper coated with silicone or a fluoropolymer. For the preparation of transdermal solutions or gels, water or organic solvents or mixtures thereof may be used. Transdermal gels may furthermore contain one or more suitable gelling agents or thickeners such as silicone, tragacanth, starch or starch derivatives, cellulose or cellulose derivatives or polyacrylic acids or derivatives thereof. Transdermal formulations may also suitably contain one or more substances that enhance absorption though the skin, such as bile salts or derivatives thereof and/or phospholipids. Transdermal formulations may be prepared according to a method disclosed in, e.g., B W Barry, “Dermatological Formulations, Percutaneous Absorption”, Marcel Dekker Inc., New York—Basel, 1983, or Y W Chien, “Transdermal Controlled Systemic Medications”, Marcel Dekker Inc., New York—Basel, 1987.

It is currently contemplated that, in the case of oral administration, the compounds might for instance be administered in an amount of from 0.5 to 2.5 mg/Kg twice daily, calculated as TRI 50f. However, the presently described methods are not limited to administration in such quantities or regimens and includes dosages and regimens outside those described in the previous sentence.

According to a further aspect there is provided an oral pharmaceutical formulation including a product as described herein, in admixture with a pharmaceutically acceptable adjuvant, diluent or carrier.

Solid dosage forms for oral administration include capsules, tablets (also called pills), powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules and tablets, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, the oral formulations may contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g., sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g., chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The active compounds may also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilising agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavouring and perfuming agents. Suspensions, in addition to the active compounds, may contain suspending agents such as ethoxylated isostearyl alcohols, polyoxyethylene sorbitol and sorbitan esters, microcrystalline cellulose, aluminium metahydroxide, bentonite, agar-agar, and tragacanth and mixtures thereof.

The presently disclosed product may be presented as solids in finely divided solid form, for example they may be micronised. Powders or finely divided solids may be encapsulated.

The active compound may be given as a single dose, in multiple doses or as a sustained release formulation.

The active compound, especially when a base addition salt or sugar derivative, may be administered as a drinking solution or drinking suspension.

Thus an orally administered compound may be presented as reconstitutable formulations, in particular in a form for reconstitution before administration as a liquid and often as drink, for example as an effervescent tablet or in particulate form (as a powder or granules). Soluble compounds may be packaged as a powder or granules for direct dissolution in the mouth; additionally contemplated are “fast melt” or “fast dissolving” oral formulations, which dissolve or disintegrate rapidly when taken into the mouth. The formulations described in the preceding two sentences may also be regarded as reconstitutable formulations in that they are reconstituted in the mouth, prior to the reconstituted formulation reaching the stomach. All these reconstitutable formulations avoid the delays associated with active ingredients in tablets or capsules reaching the blood, as a result of time taken for the tablet/capsule to disintegrate and for its contents to dissolve. Another potential benefit of reconstitutable formulations relates to active ingredients whose required dosage is too high to be incorporated in a single tablet or capsule: the ingestion of multiple tablets or capsules is considered undesirable by patients and might create an additional risk of variation in bioavailability, and the replacement of multiple tablets or capsules with a reconstitutable formulation will avoid these particular shortcomings.

A common form of dosage for the oral administration of drugs is that of particulate formulations contained in dispensing containers, e.g. sachets, particularly monodose sachets. The contents of the dispensing container are usually (but not always) poured out in, for example, a glass of water or in fruit juice or in milk, for drinking by the patient. It is helpful for the particulate formulation to be reconstituted into a drink-size volume (e.g. 50-150 ml, by way of non-limiting example).

The dispensing container may in principle be any container which may be opened to release a single dose or a part of a dose. In many instances, a container will be a single dose or monodose container, in which the container contains the correct amount for a single administration of the formulation. Alternatively, the formulation may be presented as a divided dose, in which there is provided a unit dosage which is possibly smaller than some patients require; in this latter case, the patient will take two or more unit doses in a single administration of the formulation. The dispensing container may alternatively be a metered dose container, in which a unit of formulation is metered from a reservoir of the formulation. As an alternative to a sachets, the container may be a plastics container, for example.

Thus, an exemplary unit dosage form for particulate formulations, e.g. for reconstitution as a drink, is the sachet. A sachet is a pouch formed by folding and/or sealing together the edges of a suitable material.

Particulate formulations suitable for filling into sachets may contain (but are not required to contain) diluents, flow aids, lubricants, buffering agents, granulating agents, disintegrants, solubilising agents, viscosity enhancers, sweeteners and flavours, in addition to the active ingredient. Usually, they contain anti-microbial preservations.

Effervescent tablets are another common oral dosage form and contain ingredients that react together in the presence of Water to produce carbon dioxide. The liberation of carbon dioxide when effervescent tablets are added to water promotes their disintegration and the dissolution or dispersion of the active ingredients and other components. Effervescent tablets are usually intended to be added to water to produce a solution or dispersion for oral administration. They are usually much bigger than other tablets because they are used for drugs whose dosage is large and they often contain relatively large amounts of flavouring agents.

The effervescent tablet consists of at least three components: the active ingredient; an acid; and an alkali compound (basic ingredient) constituted by a carbonate or a bicarbonate.

The acid and the alkali are the essential components which provide the effervescence and the disintegration of the tablet when it is contacted with water. As acidic component citric acid in anhydrous form is often used, but other edible acids like tartaric, fumaric, adipic and malic acid can be used as well. The carbonate, which represents a source of carbon dioxide which generates the effervescence, generally is a water-soluble alkaline carbonate. Sodium bicarbonate is one of the most used carbonates because it is very soluble and of low cost. Alternatively, modified sodium bicarbonate can be used, obtained by heating common sodium bicarbonate in order to convert the surface of its particles to sodium carbonate so increasing its stability.

Other physiologically acceptable alkaline or alkaline earth metal carbonates may be used, such as potassium or calcium (bi)carbonate, sodium carbonate, or sodium glycine carbonate.

Conventional excipients such as diluents, ligands, buffers, anti-microbial preservatives, sweeteners, flavours, colours, solubilisers, disintegrants, wetting agents and other excipients of common use may be added to the formulation. Effervescent tablets are convenient, attractive, easy to use premeasured dosage forms. These advantages, however, are balanced by hygroscopicity, which usually means that the tablets have to be manufactured in conditions of low relative humidity and packaged in containers that provide good protection against the ingress of water vapour.

“Fast melt” formulations, i.e. rapidly disintegrating or dissolving solid dose oral formulations, are described in U.S. Pat. No. 5,607,697, WO 98/46215, WO 98/14179, U.S. Pat. No. 5,871,781, U.S. Pat. No. 5,869,098 and U.S. Pat. No. 5,464,632, for example. They are also described in U.S. Pat. No. 4,642,903, U.S. Pat. No. 5,188,825, U.S. Pat. No. 5,631,023 and U.S. Pat. No. 5,827,541.

### EXAMPLES

### Examples 1 to 4

Introductory Remarks

Apparatus

Throughout the following procedures of Examples 1 to 4, standard laboratory glassware and, where appropriate, specialised apparatus for handling and transferring of air sensitive reagents are used.

All glassware is heated at 140-160° C. for at least 4 hours before use and then cooled either in a desiccator or by assembling hot and purging with a stream of dry nitrogen.

Solvents

The organic solvents used in the procedures of Examples 1 to 4 are all dry. Suitably, they are dried over sodium wire before use.

Dryness

In the drying procedures of Example 1 to 4, products are tested for dryness (including dryness in terms of organic solvent) by observing weight loss on drying. The following procedure was followed to determine loss on drying: a sample was placed in a vacuum drier and dried at 40° C. at 100 mbar for 2 hours. Products are considered dry when the decrease in weight upon drying is less than 0.5% of the total weight of the starting material.

Examples 1 to 4 describe performance of the following reaction scheme and conversion of the resultant TRI 50f to sodium and calcium salts thereof: 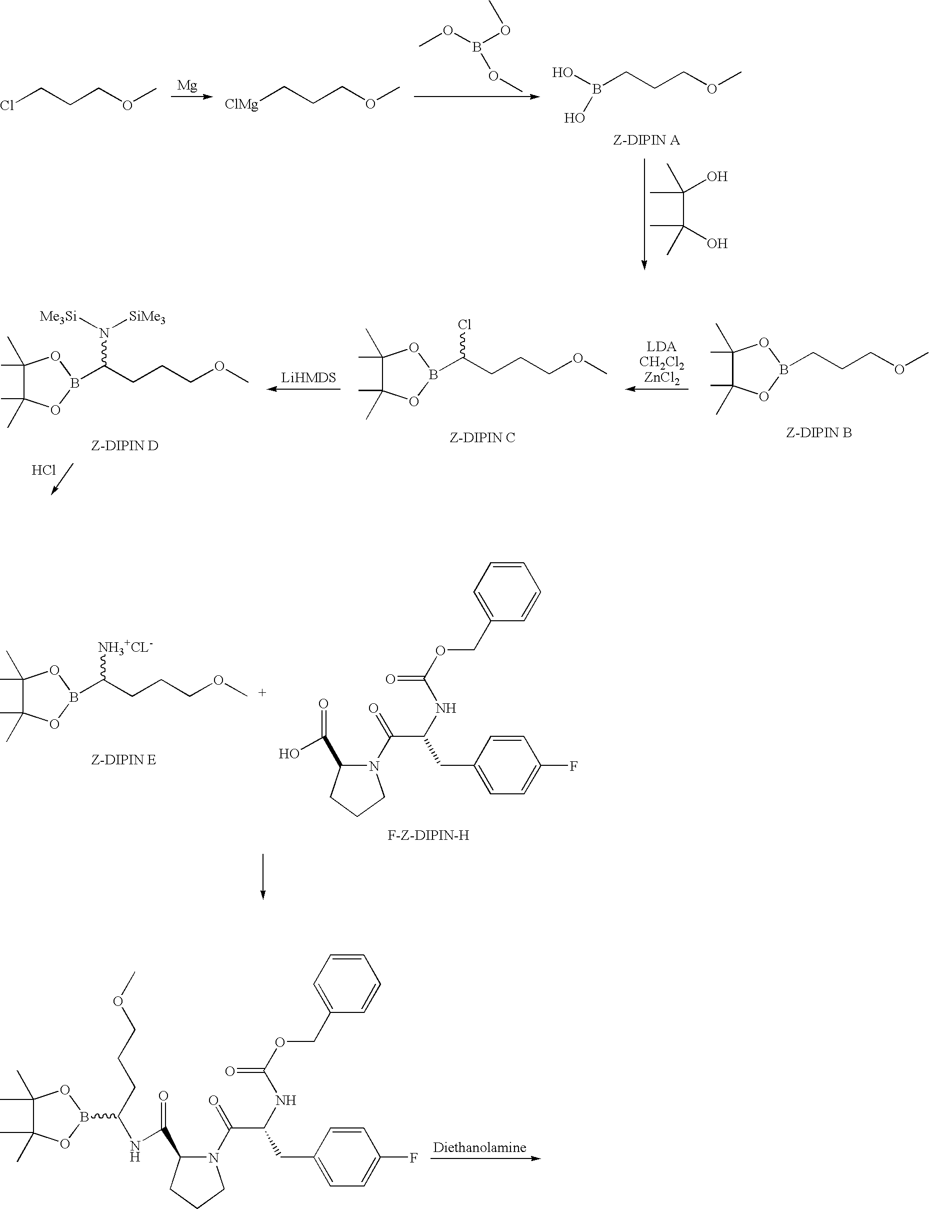 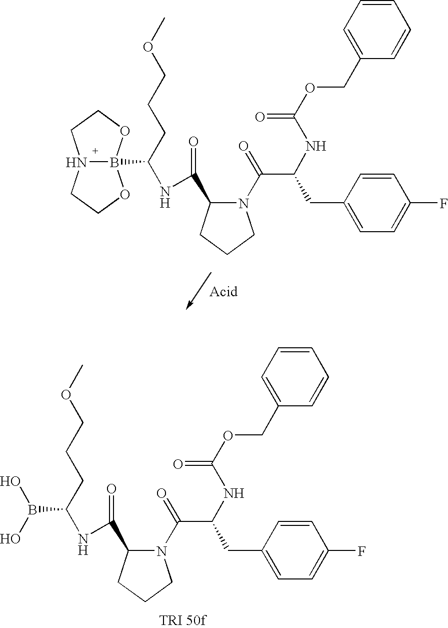
LDA=lithium diisopropylamide
LiHMDS=lithium hexamethyldisilazane, also known as lithium bis(trimethylsilyl)amide

### Example 1

Synthesis of TRI 50f Pinacol Ester

Step 1: Z-DIPIN B

Procedure A

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux until all the magnesium is consumed. After the reaction is finished, the reaction mixture is cooled to ambient temperature and slowly added to a solution of 64.4 g (620 mmole) trimethylborate in 95 ml dry tetrahydrofuran; the latter solution is cooled to below 0° C. and, if it warms up during the course of the reaction, the reaction mixture must be added to it sufficiently slowly to maintain the temperature of this solution below 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The layers are allowed to settle and the phases are separated. The aqueous layer is rewashed three times with 200 ml tert.-butylmethylether. The combined organic layers are allowed to settle and additional water separated from this solution is removed. The organic layer is dried over magnesium sulfate, filtered and evaporated to dryness. The evaporation residue is filtered from the precipitated solid and the filtrate dissolved in 175 ml toluene. 34.8 g (292 mmole) pinacol is charged to the solution followed by stirring at ambient temperature for not less than 10 hours. The solution is evaporated to dryness, dissolved in 475 ml n-heptane and washed three times with 290 ml saturated aqueous solution of sodium hydrogen carbonate. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70%) Z-DIPIN B (oil)

Procedure B

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux. After the reaction is finished, the reaction mixture is cooled to ambient temperature and slowly added to a solution of 64.4 g (620 mmole) trimethylborate in 95 ml dry tetrahydrofuran, maintaining the temperature of this solution below minus 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The organic solvent is removed by distillation under vacuum. 300 ml n-heptane is charged to the aqueous solution of the evaporation residue followed by addition of 34.8 g (292 mmole) pinacol. The two-phase-mixture is stirred at ambient temperature for not less than 2 hours. After allowing the layers to settle, the aqueous phase is separated. 300 ml n-heptane is charged to the aqueous solution and the two-phase-mixture is stirred at ambient temperature for not less than 2 hours. After allowing the layers to settle, the aqueous phase is separated. The organic layers are combined and washed once with 200 ml water, followed by 200 ml saturated sodium hydrogen carbonate solution and two further washes with 200 ml water each. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70-85%) Z-DIPIN B (oil)

Step 2: Z-DIPIN C

16.6 g (164 mmole) diisopropylamine and 220 ml tetrahydrofuran are charged and cooled to −30 to −40° C. To this solution 41.8 g (163 mmole) n-butyl lithium, 25% in n-heptane is added, followed by stirring at 0 to −5° C. for one hour. This freshly prepared solution of lithium diisopropylamide is cooled to −30° C. and then added to a solution of 27.9 g (139 mmole) Z-DIPIN B in 120 ml tetrahydrofuran and 35.5 g (418 mmole) dichloromethane at a temperature between −60 and −75° C. The solution is stirred at that temperature for half an hour followed by addition of 480 ml (240 mmole) 0.5N anhydrous Zinc(II)-chloride in tetrahydrofuran or 32.5 g (240 mmole) anhydrous solid Zinc(II)-chloride. After stirring at −65° C. for one hour, the reaction mixture is allowed to warm to ambient temperature and stirred for another 16-18 hours. The reaction mixture is evaporated to dryness (i.e. until solvent is removed) and followed by addition of 385 ml n-heptane. The reaction mixture is washed with 150 ml 5% sulfuric acid, with 190 ml saturated sodium hydrogen carbonate solution, and 180 ml saturated sodium chloride solution. The organic layer is dried over magnesium sulfate, filtered and evaporated to dryness (i.e. until solvent is removed). The oily residue is transferred into the next step without further purification.

Yield: 19 g (55%) Z-DIPIN C

Step 3: Z-DIPIN D

To a solution of 23.8 g (148 mmole) hexamethyldisilazane in 400 ml tetrahydrofuran at −15° C. is added 34.7 g (136 mmole) n-butyl lithium, 25% in n-heptane and stirred for one hour. The solution is cooled to −55° C. followed by the addition of 30.6 g (123 mmole) Z-DIPIN C dissolved in 290 ml tetrahydrofuran and 35 ml tetrahydrofuran to this freshly prepared solution of LiHMDS. The solution is allowed to warm to ambient temperature and stirred for 12 hours. The reaction mixture is evaporated to dryness, the evaporation residue dissolved in 174 ml n-heptane, washed with 170 ml water and 75 ml saturated sodium chloride solution. The organic phase is dried over magnesium sulfate, filtered and evaporated to complete dryness (i.e. until solvent is removed). The oily residue is dissolved in 100 g n-heptane. This solution is carried over into the next step without further purification.

Yield: 32.2 g (70%) Z-DIPIN D

Step 4: Z-DIPIN E

A solution of 26.6 g (71 mmole) Z-DIPIN D in 82.6 g n-heptane is diluted with 60 ml n-heptane and cooled to −60° C. followed by introduction of 10.5 g (285 mmole) hydrogen chloride. The reaction mixture is subsequently evacuated and flushed with nitrogen, while the temperature is increased in increments of about 20° C. to ambient temperature. The solvent is removed from the oily precipitate and replaced several times by 60 ml fresh n-heptane. The compound is dissolved in 60 ml tetrahydrofuran (Solution A).

Step 5 TRI 50f Pinacol Ester

Z-DIPIN E is coupled with Z-D-4-F-Phe-Pro. The procedure described in the next paragraph is generally followed.

To a different flask 130 ml tetrahydrofuran, 24.5 g (61.5 mmole) Z-D-4-F-Phe-Pro and 6.22 g (61.5 mmole) N-methylmorpholine are charged and cooled to −20° C. To this solution a solution of 8.4 g (61.5 mmole) isobutylchloroformate in 20 ml tetrahydrofuran is added and stirred for 30 minutes, followed by addition of Solution A at −25° C. Upon complete addition, up to 16 ml (115 mmole) triethylamine is added to adjust the pH to 9-10, measured using a pH stick. The reaction mixture is allowed to warm to ambient temperature and stirred for 3 hours, still under nitrogen. The solvent is evaporated to dryness and the evaporation residue dissolved in 340 ml tert.-butylmethylether (t-BME). The solution of TRI 50f pinacol ester in t-BME is washed twice with 175 ml 1.5% hydrochloric acid. The combined acidic washes are given a rewash with 175 ml t-BME. The combined organic layers are washed with 175 ml water, with 175 ml saturated sodium hydrogen carbonate solution, with 175 ml 25% sodium chloride solution, dried over magnesium sulfate and filtered. This solution is carried over into the next step without further purification.

### Example 2

Synthesis of TRI 50f Diethanolamine Adduct

The procedure described in the next paragraph is generally followed.

The starting material used in this Example is the solution of TRI 50f pinacol ester obtained in Example 1. The solution is carried forward to the synthesis of TRI 50d without further purification. The solution of TRI 50S pinacol ester in t-BME is evaporated to dryness and the evaporation residue dissolved in 80 ml diethylether. 1.25 equivalents of diethanolamine is added (based on HPLC determination of R,S,R TRI 50f pinacol ester) and the mixture heated at reflux for at least 10 hours, during which process the product precipitates. The suspension is cooled to 5-10° C., filtered and the filter residue washed with diethylether.

To improve chiral and chemical purity the wet filter cake is dissolved in 7 ml dichloromethane, cooled to 0-5° C. and the product precipitated by addition of 42 ml diethylether and filtered. The isolated wet product is dried at 35° C. in vacuum or at least 4 hours, until day.

### Example 3

Preparation of TRI 50f

Commercially available reagents and solvents (HPLC grade) were used without further purification.

Purification of compounds by preparative HPLC was performed on Gilson systems using reverse phase ThermoHypersil-Keystone Hyperprep HS C18 columns (12 μm, 100×21.2 mm), gradient 20-100% B (A=water/0.1% TFA, B=acetonitrile/0.1% TFA) over 10 min, flow=30 ml/min, injection solvent 2:1 DMSO:acetonitrile (1.6 ml), UV detection at 215 nm.

¹H NMR spectra were recorded on a Bruker 400 MHz AV spectrometer in deuterated solvents. Chemical shifts (δ) are in parts per million and coupling constants are expressed in Hz. Thin-layer chromatography (TLC) analysis was performed with Kieselgel 60 F₂₅₄ (Merck) plates and visualized using UV light.

Analytical HPLC-MS was performed on Agilent HP1100, Waters 600 or Waters 1525 LC systems using reverse phase Hypersil BDS C18 columns (5 μm, 2.1×50 mm), gradient 0-95% B (A=water/0.1% TFA, B=acetonitrile/0.1% TFA) over 2.10 min, flow=1.0 ml/min. UV spectra were recorded at 215 nm using a Gilson G1315A Diode Array Detector, G1214A single wavelength UV detector, Waters 2487 dual wavelength UV detector, Waters 2488 dual wavelength UV detector, or Waters 2996 diode array UV detector. Mass spectra were obtained over the range m/z 150 to 850 at a sampling rate of 2 scans per second or 1 scan per 1.2 seconds using Micromass LCT with Z-spray interface or Micromass LCT with Z-spray or MUX interface. Data were integrated and reported using OpenLynx and OpenLynx Browser software.

This example describes the preparation of 11 mg TRI 50f by the following scheme:

The diethanolamine precipitation step of this example (stage 5) favours precipitation of the R,S,R isomer over the R,S,S isomer. When this technique is applied to the disclosed compounds, therefore, the major product will be the R,S,R isomer. When carried out at a small scale in which preparative-HPLC is used in purification, the R,S,R isomer will be represented by the largest area under the HPLC trace. In any event, the (most) active isomer may be identified by testing the isomers for their Ki values against thrombin. In larger scale synthesis, it is convenient to resolve the two isomers by the use of diethanolamine to selectively precipitate out the R,S,R isomer to a high degree of chiral purity.

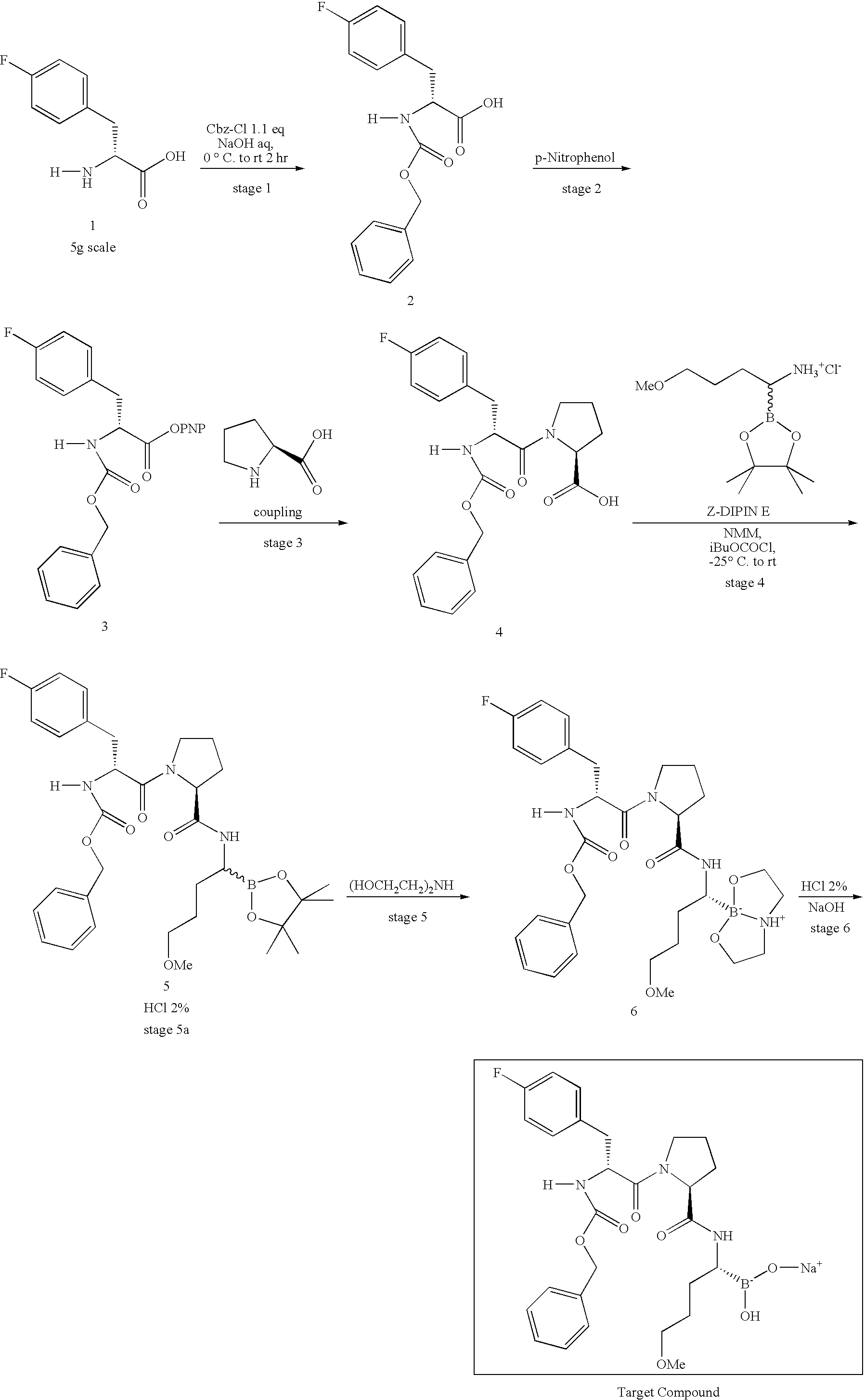

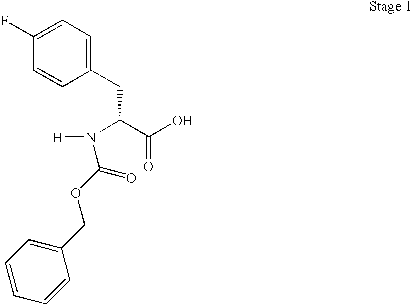

N-Cbz protected amino acid was prepared according to the literature procedure (Helv. Chim. Acta, 1973, 56, 1838-1845). The starting material 1 was obtained from a commercial source.

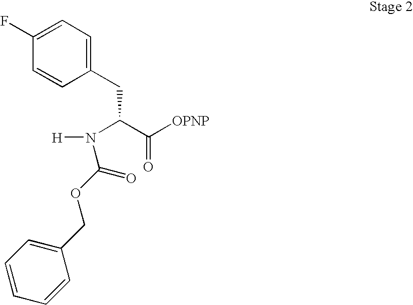

Stage 2 was carried out as described for Example 4, stage 2.

Yield: 5.06 g (73%)

Mass spectrum (ES-MS (+ve) 439 [M+H]⁺, Retention time 1.66 min

¹H-NMR (CDCl₃, 400 MHz): δ 8.30 (2H, d, Ar), 7.35-7.00 (11H, m, Ar), 5.55 (1H, br, NH), 5.15 (2H, s, OCH₂), 4.85 (1H, q, NCH), 3.20 and 3.14 (2H, m, CH₂Ph).

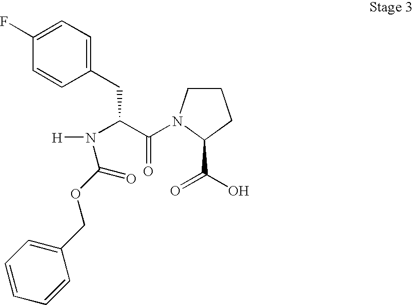

Stage 3 was carried out as described in Example 4, stage 3.

Yield: 1.31 g (46%)

Mass spectrum (ES-MS (+ve) 415 [M+H]⁺, Retention time 1.34 min

¹H-NMR (CDCl₃, 400 MHz): δ 7.35-6.90 (9H, m, Ar), 5.65 (1H, d, NH), 5.15 and 5.05 (2×1H, 2×d, OCH₂), 4.70 (1H, m, NCH), 4.35 (1H, m, NCH), 3.60 and 2.75 (2×1H, m, NCH₂), 3.0 (2H, m, CH₂Ph), 2.20-1.65 (4H, m, alkyl).

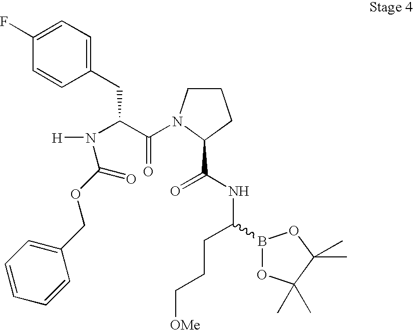

Stage 4 was carried out as described in Example 4, stage 4.

Crude Yield: 272 mg (60%)

Mass spectrum (ES-MS (+ve) 526 [M-(pinacol and water)]+H]⁺, Retention time 1.26. (ES-MS (+ve)) 626 [M+H]⁺, Retention time 1.47.

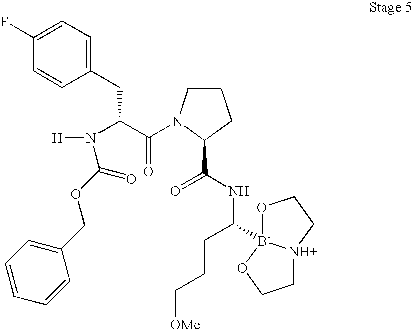

Stage 5 was carried out as described in Example 4, stage 5.

Mass spectrum (ES-MS (+ve) 526 [M-(diethanolamine and water)]+H]⁺, Retention time 1.26.

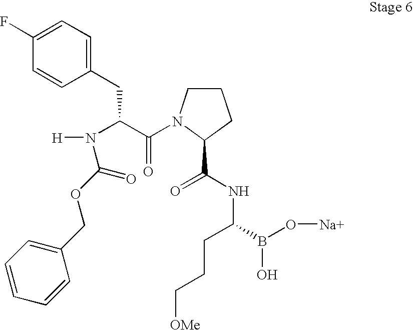

Stage 6 was carried out as described in Example 4, stage 6.

Yield: 11 mg

Mass spectrum (ES-MS (+ve) 526 [M-water]+H]⁺, Retention time 6.66.

¹H-NMR (CDCl₃, 400 MHz): Due to restricted rotation ¹H NMR was broad and difficult to assign. δ 7.35-6.90 (9H, m, Ar), 5.4 (m), 5.2-4.8 (m), 3.6 (m), 3.35-3.10 (m), 3.00-2.85 (m), 2.70-2.50 (m), 2.2-1.40 (m).

The monosodium salt was prepared as for Example 4, stage 5 isomer 1.

### Example 5

Preparation of Sodium Salt of TRI 50f

The following procedure is generally followed.

2.5 mole TRI 50f diethanolamine ester from Example 2 is dissolved in 10.5 L dichloromethane. 11 L 2% hydrochloric acid is added and the mixture is stirred for at most 30 minutes (optimally about 20 minutes) at room temperature. A precipitate forms in the organic phase. After stirring, the layers are allowed to settle and separated. The aqueous layer is rewashed twice with 2.2 L dichloromethane. The combined organic layers are washed with a solution of 625 g ammonium chloride in 2.25 L water. (The ammonium chloride buffers the pH of the aqueous extractions to be within a range of from about pH 1-2 to about pH 4-5, as strongly acidic conditions might cleave peptide bonds). The organic phase is dried over magnesium sulfate, filtered and the filtrate evaporated to dryness. An assay of the free boronic acid is performed for at most 30 mins at room temperature and the amounts of the solvents and base for conversion of the acid to the salt are calculated. If 2.5 mol of the free acid is obtained, the evaporation residue is dissolved in 5 L acetonitrile followed by addition of a solution of 100 g (2.5 mole) sodium hydroxide as a 5% solution in 2.2 L water. The solution is stirred for two hours at ambient temperature (e.g. 15-30° C., optimally room temperature) and then evaporated in vacuum (of ca. 10 mmHg) at a temperature not exceeding 35° C. The evaporation residue is repeatedly dissolved in 3.5 L fresh acetonitrile and evaporated to dryness to remove traces of water. If the evaporation residue is dry, it is dissolved in 3 L acetonitrile (or alternatively in 6 L THF) and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum at a temperature initially of about 10° C. and then increasing to a limit of about 35° C., until dry.

### Example 6

Preparation of Calcium Salt of TRI 50f

The following procedure is generally followed.

2.5 mole TRI 50f pinacol ester from Example 2 is dissolved in 10.5 L dichloromethane. 11 L 2% hydrochloric acid is added and the mixture is stirred for at most 30 minutes (optimally about 20 minutes) at room temperature. After stirring the layers are allowed to settle and separated. The aqueous layer is rewashed twice with 2.2 L dichloromethane. The combined organic layers are washed with a solution of 625 g ammonium chloride in 2.25 L water. The organic phase is dried over magnesium sulfate, filtered and the filtrate evaporated to dryness. An assay of the free boronic acid is performed and the amounts of the solvents and base for conversion of the acid to the salt are calculated. If 2.5 mol of the free acid is obtained, the evaporation residue is dissolved in 5 L acetonitrile followed by addition of a suspension of 93 g (1.25 mole) calcium hydroxide in 1 L water. The solution is stirred for two hours at ambient temperature (e.g. 15-30° C., optimally room temperature) and then evaporated under vacuum (of ca. 10 mmHg) at a temperature initially of about 10° C. and then increasing to a limit of about 35° C. The evaporation residue is repeatedly dissolved in 3.5 L fresh acetonitrile and evaporated to dryness to remove traces of water. If the evaporation residue is dry, it is dissolved in 6 L tetrahydrofuran and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum (of ca. 10 mmHg) at a temperature below 35° C. until dry.

The procedures of Examples 1 to 4 may be scaled up and, if operated carefully, will produce highly pure salts. In the diethanolamine precipitation step it is important to use 1.25 equivalents of diethanolamine per equivalent of (R,S,R) TRI 50f pinacol ester. In the hydrolysis of the diethanolamine ester, it is important to avoid excessively long contact with the aqueous acid. Likewise the TRI 50f pinacol ester should be synthesised via the Grignard reaction to Z-DIPIN A.

### Example 7

Preparation of Lithium Salt of TRI 50f

The following procedure is generally followed.

TRI 50f (38.1 mM) is dissolved in acetonitrile (200 ml) with stirring at room temperature. To this solution is added LiOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water necessary with light warming for about 20 minutes. The solution is filtered through filter paper and evacuated to dryness, again with the temperature of the solution not exceeding 37° C. The resultant product is dried under vacuum overnight to normally yield a white brittle solid.

The salt is then dried under vacuum over silica to constant weight (72 h).

### Example 8

Preparation of Zinc Salt of TRI 50f

The following procedure is generally followed.

The relative solubility of zinc hydroxide is such that, if the hydroxide had been used to prepare the corresponding TRI 50f salt using the procedure of Example 4, it would not have resulted in homogeneous salt formation. The following procedure is generally followed to prepare the zinc salt.

TRI 50f sodium salt (4.10 mM) is dissolved in distilled water (100 ml) at room temperature and zinc chloride in THF (4.27 ml, 0.5M) is carefully added with stirring. A white precipitate that immediately formed is filtered off and washed with distilled water. This solid is dissolved in ethyl acetate and washed with distilled water (2×50 ml). The organic solution is evacuated to dryness and the white solid produced dried over silica in a desiccator for 3 days before microanalysis.

### Example 9

Preparation of Magnesium Salt of TRI 50f

The following procedure is generally followed.

TRI 50f (1.90 mM) is dissolved in methanol (10 ml) and stirred at room temperature. To this solution is added magnesium methoxide (Mg(CH₃O)₂) in methanol (1.05 ml, 7.84 wt %). This solution is stirred for 2 hours at room temperature filtered and evacuated to 5 ml. Water (25 ml) is then added and the solution evacuated down to dryness to yield a white solid. This is dried over silica for 72 hours before being sent for microanalysis

### Example 10

Determination of Diastereomeric Excess

TRI 50b, crude, contains three chiral centres. Two of them are fixed by the use of enantiomerically pure amino acids ((R)-Phe and (S)-Pro). The third one is formed during the synthesis. The favoured epimer is the desired TRI 50b, Isomer I (R,S,R-TRI 50b). Both epimers of TRI 50b are clearly baseline separated by the HPLC method, thus allowing determination of the diasteromeric excess (de) of TRI 50b.

TRI 50d is not stable under the conditions applied for HPLC purity determination, but decomposes rapidly on sample preparation to TRI 50c, so that TRI 50d and TRI 50c show the same HPLC traces.

The two isomers of TRI 50c are not baseline separated in HPLC, but both isomers are clearly visible. This becomes obvious, when TRI 50b, crude (mixture of both isomers) is converted with phenylboronic acid to TRI 50c, crude. Both isomers of TRI 50c are observed in HPLC nearly at the same relation as before in TRI 50b, crude.

Upon synthesis of TRI 50d from TRI 50b, crude, only one diastereoisomer is precipitated. In this case HPLC shows only one peak for TRI 50c, where a very small fronting is observed. Precipitation from dichloromethane/diethylether removes the fronting efficiently. The level of removal of Isomer II cannot be quantified by this HPLC method. Therefore samples before reprecipitation and after one and two reprecipitations were esterified with pinacol and the resulting samples of TRI 50b analysed by HPLC. Thus a de of 95.4% was determined for the crude sample. The reprecipitated sample resulted in a de of 99.0% and finally the sample that was reprecipitated twice showed a de of 99.5%.

These results clearly show the preferred precipitation of Isomer I, whereas Isomer II remains in solution.

It will be appreciated from the foregoing that the disclosure provides boronic acid salts useful for pharmaceutical purposes and which feature one or more of the following attributes: (1) improved hydrolytic stability; (2) improved stability against deboronation; and (3), in any event, not suggested by the prior art.

The selection of active ingredient for a pharmaceutical composition is a complex task, which requires consideration not only of biological properties (including bioavailability) but also of physicochemical properties desirable for processing, formulation and storage. Bioavailability itself is dependent on various factors, often including in vivo stability, solvation properties and absorption properties, each in turn potentially dependent on multiple physical, chemical and/or biological behaviours.

### Example 4

Process Details

The target compound is prepared by the following procedure:

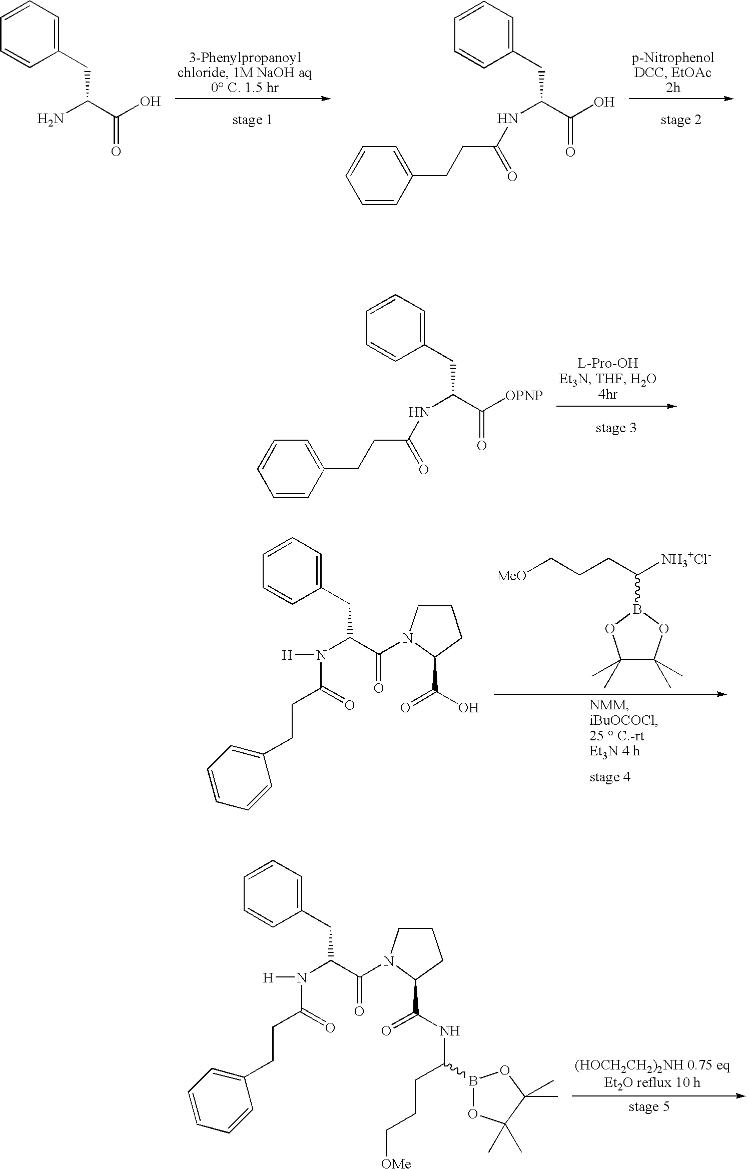

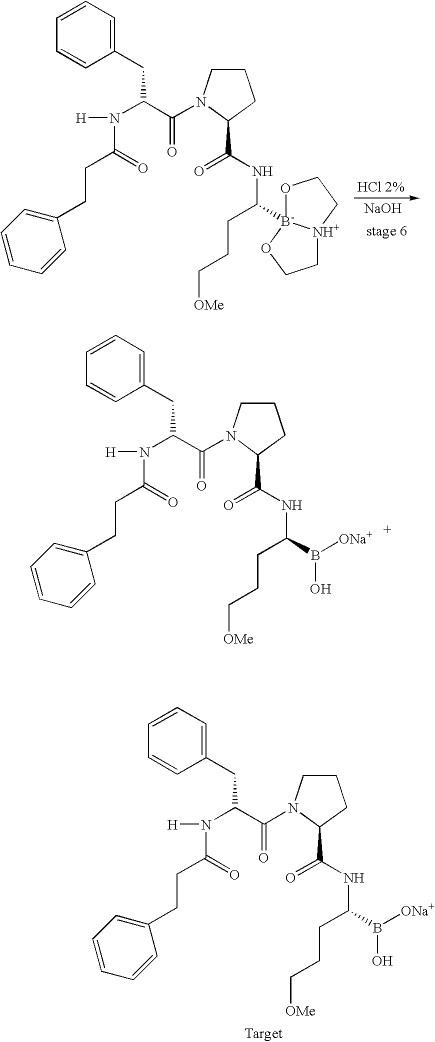

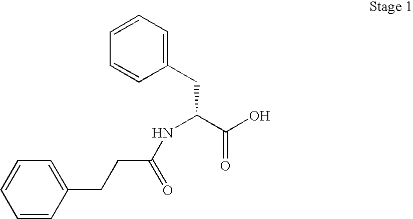

p N-(3-Phenyl-propionyl)-D-phenylalanine* was prepared according to the literature (J. Chem. Soc. Perkin Trans, 1, 1982, 2939-2948)
*D-phenylalanine is the same as (R)-phenylalanine.

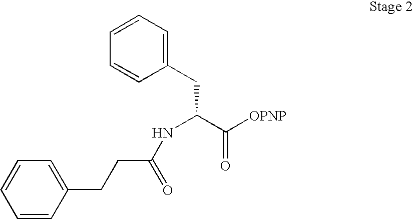

N-(3-Phenyl-propionyl)-D-phenylalanine (5 g, 16.8 mmol) and p-nitro phenol (2.69 g, 19.3 mmol) were dissolved in EtOAc (50 ml) and cooled to 0° C. To this solution 1,3-dicyclohexylcarbodiimide (3.47 g, 16.8 mmol) was added. After 30 min at 0° C. and 2 hrs at room temperature the reaction mixture was filtered off and washed with EtOAc (100 ml). Then combined liquid and washing were evaporated to dryness in vacuo and the product was purified by crystallisation from ethanol.

Yield: 5.5 g (78%)

Mass spectrum (ES-MS (+ve) 419 [M+H]⁺, Retention time 1.6 min

¹H-NMR (CDCl₃, 400 MHz): δ 8.22 (2H, d, Ar), 7.35-7.05 (12H, m, Ar), 5.90 (1H, br, NH), 5.05 (1H, q, NCH), 3.21 and 3.14 (2×1H, 2×dd, CH₂Ph), 2.97 (2H, t, PhCH₂), 2.55 (2H, m, COCH₂).

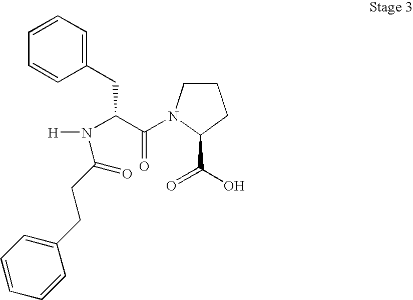

The p nitro phenyl ester of N-(3-phenyl-propionyl)-D-phenylalanine (5.0 g, 11.9 mmol) was dissolved in THF (40 ml). A solution of L-proline‡ (1.37 g, 11.9 mmol) and triethyl amine (1.20 g, 11.9 mmol) in water (20 ml) was added. The reaction mixture was stirred for 20 hrs at room temperature. THF was removed in vacuo and the aqueous layer was diluted with water then extracted with 3×EtOAc. pH of the aqueous layer was adjusted to 3 by adding 10% citric acid then again extracted with several times with EtOAc. The combined organic layers were washed with water, dried (Na₂SO₄). Solvent was removed in vacuo and the product was purified by crystallisation from EtOAc/Heptane.
‡L-proline is the same as (S)-proline

Yield: 4.1 g (85%)

Mass spectrum (ES-MS (+ve) 395 [M+H]⁺, Retention time 1.26 min

¹H-NMR (CDCl₃, 400 MHz): Due to the restricted rotation ¹H NMR was broad and difficult to assign. δ 7.32-7.13 (10H, m, Ar), 6.20 (1H, br, NH), 4.95 (1H, m, NCH), 4.10 and 4.31 (2×1H, 2×m, NCH, restricted rotation), 3.52 (2H, m), 3.10-1.50 (10H, m).

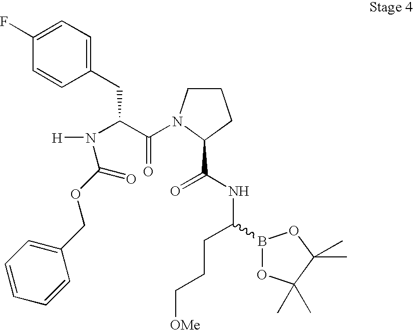

The dipeptide (0.19 g, 0.48 mmol) and N-methylmorpholine (48 mg, 0.48 mmol) were dissolved in THF (15 ml) and cooled to −20° C. under nitrogen. To this solution isobutyl chloroformate (65 mg, 0.48 mmol) was added and stirred at −20° C. for 30 min. Then Z-DIPIN-E (0.12 g, 0.536 mmol) was added in THF at −25° C. Then triethylamine (0.126 ml, 91 mmol) was added and allowed to warm to room temperature and stirred for 16 hrs. The solvent was evaporated to dryness, the residue was dissolved in TBME, then washed with 0.2M HCl, then acid layer extracted with TBME, the combined TBME layers were washed with water sat. NaHCO₃ and brine, dried (Na₂SO₄). Solvent was removed in vacuo and the crude was used next stage without further purification.

Crude Yield: 314 mg (98%)

Mass spectrum (ES-MS (+ve) 506 [M-(pinacol and water)]+H]⁺, Retention time 1.22 and 1.16 min (two diastereomers). (ES-MS (+ve) 606 [M+H]⁺, Retention time 1.47 and 1.4 min (two diastereomers).

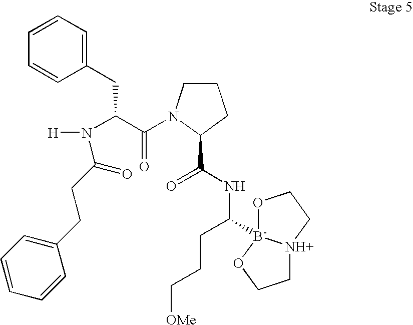

The boronate ester (0.31 g, 0.51 mmol) was dissolved in ether (8 ml) and diethanolamine (0.04 g, 0.389 mmol) added, then the reaction mixture was refluxed for 10 h, cooled to 5° C. and an oily precipitate formed, ether was decanted, the oily residue was washed with cold ether and the residue was used in the next stage without further purification.

Mass spectrum (ES-MS (+ve) 506 [M-(diethanolamine and water)]+H]⁺, Retention time 1.23 and 1.18 min (two diastereomers).

Stage 6

Diethanolamine salt (0.3 g, 0.50 mmol) was dissolved in dichloromethane (5 ml) and 2% HCl (5 ml) was added and stirred at room temperature for 30 min. The organic layer was separated, the aqueous layer washed with 2× dichloromethane. The combined organic layers were washed with ammonium chloride solution and dried (MgSO₄). Solvent was removed in vacuo and the product was purified by prep-HPLC.

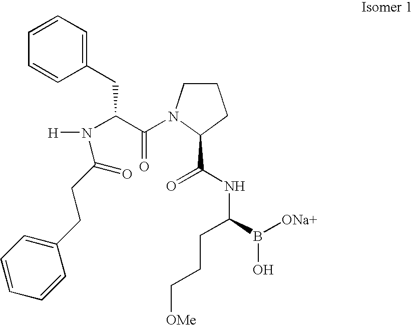

Yield: 39 mg

Mass spectrum (ES-MS (+ve) 506 [M-water]+H]⁺, Retention time 6.29 min.

¹H-NMR (CDCl₃, 400 MHz): Due to restricted rotation ¹H NMR was broad and difficult to assign. δ 7.40-7.15 (10H, m, Ar), 6.10-6.30 (1H, br, NH), 4.80 (m), 4.5 (m), 3.7 (m), 3.40-3.20 (m), 2.90-2.70 (m), 2.60-2.30 (m), 2.2-1.50 (m).

Mono Sodium Salt Formation:

Boronic acid (30 mg, 0.057 mmol) was dissolved in MeCN (0.3 ml) then 5% NaOH (4.5 μl, 0.056 mmol) was added and stirred for 2 h. Solvents were removed under flow of nitrogen and dried under high vacuum.

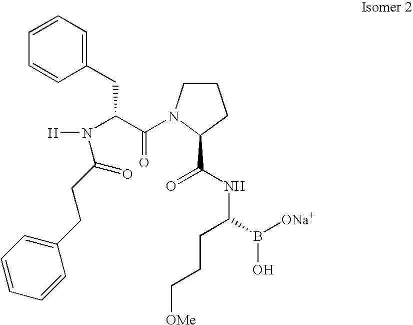

Yield: 26 mg

Mass spectrum (ES-MS (+ve) 506 [M-water]+H]⁺, Retention time 6.46 min.

¹H-NMR (CDCl₃, 400 MHz): Due to the restricted rotation ¹H NMR was broad and difficult to assign. μ 7.30-7.15 (10H, m, Ar), 6.20 and 6.30 (1H, br, NH), 4.60-4.15 (m), 3.6 (m), 3.40-3.15 (m), 2.95-2.80 (m), 2.70-2.30 (m), 2.2-1.40 (m).

The mono sodium salt was prepared as for isomer 1.

### Example 11

TRI 50f Stability in Simulated Gastric Fluid

Approximately 2.5 mg of the compound is dissolved in 50 ml gastric fluid (37° C., according to BP with pepsin, an aliquot is transferred into an HPLC-vial and analysed immediately (time period=0). The aliquots for further time periods in HPLC-vials are stored in an autosampler at 37° C. Analyses are carried out using Kromasil Kr-04 after 35, 70, 105 and 140 mins.

| | | TRIf monosodium salt | | |
|---|---|---|---|---|
| Gastric Juice (min) | rt [min]: | 14.5 | 16.0 | 17.3 |
| 0 | results | 100.0 | 100.0 | 100.0 |
| 35 | [peak-area-%]* | 89.6 | 101.4 | 99.1 |
| 70 | | 84.6 | 101.1 | 101.8 |
| 105 | | 94.2 | 97.7 | 100.3 |
| 140 | | 93.9 | 94.6 | 91.4 |
| 175 | | 94.9 | 98.6 | 97.7 |
| 210 | | 98.1 | 94.4 | 94.1 |
| concentration [μg/ml]: | | 22.8 | 22.8 | 22.8 |

| | |
|---|---|
| solvent: | water/gastric fluid |

*initial set to 100 peak-area-%

### Example 12

TRI 50f Potency and Selectivity

Method A

50 μl thrombin (33.3 ng/ml in assay buffer) and 20 μl vehicle or compound solution were added to 110 μl assay buffer (100 mM Na orthophosphate (80% Na₂HPO₄ and 20% NaH₂PO₄), 200 mM NaCl, 0.5% PEG 6000, 0.02% Na azide, pH 7.5) and incubated for 5 minutes at 37° C. After the incubation period, 20 μl of thrombin substrate (40 μM, S52238) was added and changes in Vmax monitored on a plate reader for 10 minutes using a wavelength of 405 nm at 37° C.

Method B

50 μl Plasmin (75 ng/ml in assay buffer) and 20 μl vehicle or compound solution were added to 110 μl assay buffer (100 mM Na orthophosphate (80% Na₂HPO₄ and 20% NaH₂PO₄), 200 mM NaCl, 0.5% PEG 6000, 0.02% Na azide, pH 7.5) and incubated for 5 minutes at 37° C. After the incubation period, 20 μl of plasmin substrate (7 mM, S2366) was added and changes in Vmax monitored on a plate reader for 10 minutes using a wavelength of 405 nm at 37° C.

Method C

50 μl Trypsin (20 ng/ml in assay buffer) and 20 μl vehicle or compound solution were added to 110 μl assay buffer (100 mM Na orthophosphate (80% Na₂HPO₄ and 20% NaH₂PO₄), 200 mM NaCl, 0.5% PEG 6000, 0.02% Na azide, pH 7.5) and incubated for 5 minutes at 37° C. After the incubation period, 20 μl of trypsin substrate (250 μM, S2222) was added and changes in Vmax monitored on a plate reader for 10 minutes using a wavelength of 405 nm at 37° C.

Results: Ki Values for TRI 50f

| Enzyme | IC50 (μM) | Ki (μM) | Selectivity |
|---|---|---|---|
| Thrombin | <0.1 | <0.075 | |
| Plasmin | >100 | >100 | >1000 |
| Trypsin | >10 | >5 | >100 |

The present disclosure includes the subject matter of the following paragraphs:
1. A compound selected from boronic acids of formula (I), and salts, prodrugs and prodrug salts thereof formula (I): 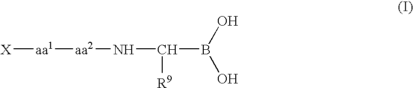
wherein
X is H (to form NH₂) or an amino-protecting group;
aa¹ is an amino acid residue having a side chain selected from formula (A) and (B):
—(CO)ₐ—(CH₂)_{b}-D_{c}-(CH₂)_{d}-E (A)
—(CO)ₐ—(CH₂)_{b}-D_{c}-Cₑ(E¹)(E²)(E³) (B)
wherein
a is 0 or 1;
e is 1;
b and d are independently 0 or an integer such that (b+d) is from 0 to 5 or, as the case may be, (b+e) is from 1 to 5;
c is 0 or 1;
D is O or S;
E is a saturated or unsaturated cyclic hydrocarbyl group which normally contains up to 14 members; and
E¹, E² and E³ are each independently selected from the group consisting of 5-6 membered saturated or unsaturated hydrocarbyl rings, or one of E¹, E² and E³ is hydrogen and the other two are a said hydrocarbyl ring,
and wherein E, E¹, E² and E³ are halogenated, a particular halogen being fluorine;
   aa² is a residue of an amino acid which binds to the thrombin S2 subsite; and
   R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ-W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms.
   2. A compound of paragraph 1 wherein R⁹ is an alkoxyalkyl group.
   3. A compound of paragraph 1 or paragraph 2 wherein R⁹ is methoxypropyl.
   4. A compound of any of paragraphs 1 to 3 wherein a and c are both 0 and (a+b+c+d) and (a+b+c+e) are 1, 2 or 3.
   5. A compound of any of paragraphs 1 to 4 wherein E, E¹, E² and E³ are each independently phenyl fluorinated at least the 4-position or fluorinated cyclohexyl, one E¹, E² and E³ optionally being H.
   6. A compound of paragraph 4 or paragraph 5 wherein aa¹ is of R configuration, aa² is of S configuration, and the fragment —NHCH(R⁹)—B(OH) is of R configuration.
   7. A compound of any of paragraphs 1 to 6 wherein the boronic acid has a Ki for thrombin of about 100 nM or less.
   8. A compound of paragraph 7 wherein the boronic acid has a Ki for thrombin of about 20 nM or less.
   9. A compound in pharmaceutical dosage selected from boronic acids of formula (II), and salts, prodrugs and prodrug salts thereof: 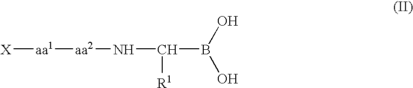
   where:
   X is H (to form NH₂) or an amino-protecting group;
   aa¹ is an amino acid having a side chain selected from:
   —CH₂-E (A′)
   —C(E¹)(E²)(E³) (B′)
where
E is a saturated or unsaturated cyclic hydrocarbyl group which normally contains up to 14 members; and
E¹, E² and E³ are each independently selected from the group consisting of 5-6 membered saturated or unsaturated hydrocarbyl rings, or one of E¹, E² and E³ is hydrogen and the other two are a said hydrocarbyl ring,
and wherein E, E¹, E² and E³ are halogenated, a particular halogen being fluorine;
   aa² is an imino acid having from 4 to 6 ring members;
   R¹ is a group of the formula —(CH₂)ₛ-Z, where s is 2, 3 or 4 and Z is —OH, —OMe, —OEt or halogen (F, Cl, Br or I).
   10. A compound of paragraph 9 wherein E, E¹, E² and E³ are each independently phenyl fluorinated at least the 4-position or fluorinated cyclohexyl, one of E¹, E² and E³ optionally being H.
   11. A compound of paragraph 9 wherein aa¹ is selected from di(4-F)-Dpa, 4-F-Phe, di(4-F)-Dcha and 4-F-Cha.
   12. A compound of any of paragraphs 9 to 11 wherein aa¹ is of R-configuration.
   13. A compound of paragraph 9 wherein aa¹ is (R)-4-F-Phe or (R)-di(4-F)-Dpa.
   14. A compound of paragraph 9 wherein aa¹ is (R)-4-F-Phe.
   15. A compound of any of paragraphs 9 to 14 wherein aa² is a residue of an imino acid of formula (IV) 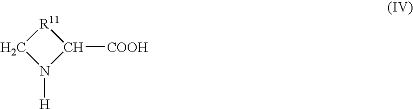
   where R¹¹ is —CH₂—, —CH₂—CH₂—, —CH═CH—, —S—CH₂—, —S—C(CH₃)₂— or —CH₂—CH₂—CH₂—, which group, when the ring is 5- or 6-membered, is optionally substituted at one or more —CH₂— groups by from 1 to 3 C₁-C₃ alkyl groups.
   16. A compound of paragraph 15 wherein aa² is of S-configuration.
   17. A compound of paragraph 15 wherein aa² is an (S)-proline residue.
   18. A compound of paragraph 9, wherein aa¹-aa² is a residue of (R)-Phe-(S)-Pro.
   19. A compound of any of paragraphs 9 to 18 wherein R¹ is 2-bromoethyl, 2-chloroethyl, 2-methoxyethyl, 3-bromopropyl, 3-chloropropyl or 3-methoxypropyl.
   20. A compound of any of paragraphs 9 to 18 wherein R¹ is 3-methoxypropyl.
   21. A compound of any of paragraphs 9 to 20 where X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by one or more (e.g. 1, 2, 3, 4 or 5) halogens (e.g. F), for example at least at the 4-position, and/or by 1, 2 or 3 substituents selected from amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group.
   22. A compound of paragraph 21 wherein said 5 to 13-membered cyclic group is aromatic or heteroaromatic.
   23. A compound of paragraph 22 wherein said 5 to 13-membered cyclic group is phenyl or a 6-membered heteroaromatic group.
   24. A compound of any of paragraphs 9 to 20 wherein X is R⁶—(CH₂)ₚ—C(O)— or R⁶—(CH₂)ₚ—O—C(O)— and p is 0, 1 or 2.
   25. A compound of any of paragraphs 9 to 20 wherein X is benzyloxycarbonyl.
   26. A compound of paragraph 9 wherein the boronic acid is of formula (VIII):
   X—(R)-4-F-Phe-(S)-Pro-(R)-Mpg-B(OH)₂ (VIII)
   27. A compound of any of paragraph 1 to 26 wherein said at least one substituent is selected from halo, hydroxy, amino, amidino, guanidino, nitro, carboxyl (whether as free acid or salt), esterified carboxyl (e.g. having C₁-C₄ alkyl or alkoxyalkyl as the ester-forming group, for example).
   28. A compound of any of paragraphs 1 to 27 which comprises the free boronic acid.
   29. A compound of any of paragraphs 1 to 27 which comprises a prodrug of the boronic acid.
   30. A compound of any of paragraphs 1 to 27 which comprises an ester of the boronic acid.
   31. A compound of paragraph 30 wherein the ester is with a diol.
   32. A compound of paragraph 31 wherein the diol is pinacol, pinanediol, diethanolamine, neopentylglycol, 1,2-ethanediol, 1,2-propanediol, 1,3-propanediol, 2,3-butanediol, 1,2-diisopropylethanediol, 5,6-decanediol or 1,2-dicyclohexylethanediol.
   33. A compound of paragraph 31 wherein the diol is a sugar.
   34. A compound of paragraph 33 wherein the sugar is a monosaccharide or a disaccharide.
   35. A compound of paragraph 33 or 34 wherein the sugar is a reduced sugar.
   36. A compound of paragraph 35 wherein the sugar is mannitol or sorbitol.
   37. A compound of any of paragraphs 1 to 32 which comprises a reaction product of the boronic acid and a pharmaceutically acceptable base.
   38. A compound of any of paragraphs 1 to 27 which comprises a pharmaceutically acceptable base addition salt of the boronic acid.
   39. A compound of paragraph 38 wherein the salt comprises boronate ions derived from the boronic acid and monovalent counter-ions.
   40. A compound of paragraph 38 or paragraph 39 which comprises a salt of the peptide boronic acid with an alkali metal or a strongly basic organic nitrogen-containing compound.
   41. A compound of paragraph 40 wherein the strongly basic organic nitrogen-containing compound is a guanidine, a guanidine analogue or an amine.
   42. A compound of any of paragraphs 38 to 44 wherein the salt comprises a salt of the boronic acid with a metal.
   43. A compound of paragraph 38 wherein the salt comprises a salt of the boronic acid with an alkali metal, an aminosugar, a guanidine or an amine of formula (XI): 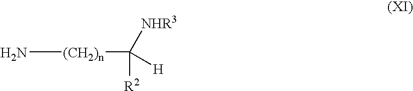
   where n is from 1 to 6, R² is H, carboxylate or derivatised carboxylate, R³ is H, C₁-C₄ alkyl or a residue of a natural or unnatural amino acid.
   44. A compound of paragraph 38 wherein the salt comprises a salt of the boronic acid with a guanidine or with an amine of formula (XI): 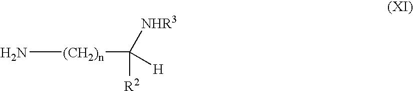
   where n is from 1 to 6, R² is H, carboxylate or derivatised carboxylate, R³ is H, C₁-C₄ alkyl or a residue of a natural or unnatural amino acid.
   45. A compound of paragraph 44 which comprises a guanidine salt of the boronic acid.
   46. A compound of paragraph 45 which comprises a salt of the boronic acid with L-arginine or an L-arginine analogue.
   47. A compound of paragraph 46 wherein the L-arginine analogue is D-arginine, or the D- or L-isomers of homoarginine, agmatine[(4-aminobutyl)guanidine], NG-nitro-L-arginine methyl ester, or a 2-amino pyrimidines.
   48. A compound of paragraph 45 which comprises a salt of the boronic acid with a guanidine of formula (VII) 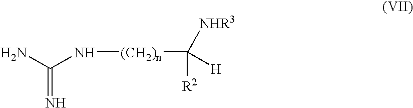
   where n is from 1 to 6, R² is H, carboxylate or derivatised carboxylate, R³ is H, C₁-C₄ alkyl or a residue of a natural or unnatural amino acid.
   49. A compound of paragraph 48, wherein n is 2, 3 or 4.
   50. A compound of paragraph 48 or paragraph 49 where the derivatised carboxylate forms a C₁-C₄ alkyl ester or amide.
   51. A compound of any of paragraphs 48 to 50 wherein the compound of formula (VII) is of L-configuration.
   52. A compound of paragraph 45 which comprises an L-arginine salt of the peptide boronic acid.
   53. A compound of paragraph 44 which comprises a salt of the boronic acid with an amine of formula (IX).
   54. A compound of paragraph 53, wherein n is 2, 3 or 4.
   55. A compound of paragraph 53 or paragraph 54 where the derivatised carboxylate forms a C₁-C₄ alkyl ester or amide.
   56. A compound of any of paragraphs 53 to 55 wherein the amine of formula (IX) is of L-configuration.
   57. A compound of paragraph 53 which comprises an L-lysine salt of the boronic acid.
   58. A compound of paragraph 38 wherein the salt comprises an alkali metal salt of the boronic acid.
   59. A compound of paragraph 58 wherein the alkali metal is potassium.
   60. A compound of paragraph 58 wherein the alkali metal is sodium.
   61. A compound of paragraph 58 wherein the alkali metal is lithium.
   62. A compound of paragraph 38 wherein the salt comprises an aminosugar salt of the boronic acid.
   63. A compound of paragraph 62 wherein the aminosugar is a ring-opened sugar.
   64. A compound of paragraph 63 wherein the aminosugar is a glucamine.
   65. A compound of paragraph 62 wherein the aminosugar is a cyclic aminosugar.
   66. A compound of any of paragraphs 62 to 65 wherein the aminosugar is N-unsubstituted.
   67. A compound of any of paragraphs 62 to 65 wherein the aminosugar is N-substituted by one or two substituents.
   68. A compound of paragraph 67 wherein the or each substituent is a hydrocarbyl group.
   69. A compound of paragraph 67 wherein the or each substituent is selected from the group consisting of alkyl and aryl moieties.
   70. A compound of paragraph 69 wherein the or each substituent is selected from the group consisting of C₁, C₂, C₃, C₄, C₅, C₆, C₇ and C₈ alkyl groups
   71. A compound of any of paragraphs 67 to 70 wherein there is a single N-substituent.
   72. A compound of paragraph 62 wherein the glucamine is N-methyl-D-glucamine.
   73. A compound of any of paragraphs 38 to 72 which comprises boronate ions derived from the peptide boronic acid and has a stoichiometry consistent with the boronate ions carrying a single negative charge.
   74. A compound of any of paragraphs 38 to 72 wherein the salt consists essentially of acid salt (that is, wherein one B—OH group remains protonated).
   75. A compound of any of paragraphs 38 to 74 wherein the salt comprises a boronate ion derived from the peptide boronic acid and a counter-ion and wherein the salt consists essentially of a salt having a single type of counter-ion.
   76. A product for use as a pharmaceutical, comprising a compound of any of paragraphs 1 to 75.
   77. A pharmaceutical formulation comprising a compound of any of paragraphs 1 to 71.
   78. A pharmaceutical formulation of paragraph 77 which is adapted for intravenous administration.
   79. A pharmaceutical formulation of paragraph 77 which is adapted for oral administration.
   80. A method of inhibiting thrombin in the treatment of disease comprising administering to a mammal a therapeutically effective amount of a compound of any of paragraph 1 to 75.
   81. The use of a compound of any of paragraph 1 to 75 for the manufacture of a medicament for treating thrombosis.
   82. A method of treating venous and/or arterial thrombosis by prophylaxis or therapy, comprising parenterally administering to a mammal suffering from, or at risk of suffering from, arterial thrombosis a therapeutically effective amount of a product selected form the compounds defined any of paragraphs 1 to 75.
   83. A method of paragraph 82 wherein the disease is an acute coronary syndrome.
   84. A method of paragraph 82 wherein the disease is acute myocardial infarction.
   85. A method of paragraph 82 wherein the disease is a venous thromboembolic event, selected from the group consisting of deep vein thrombosis and pulmonary embolism.
   86. A method for preventing thrombosis in a haemodialysis circuit of a patient, comprising parenterally administering to the patient a therapeutically effective amount of a product selected from the compounds defined any of paragraphs 1 to 75.
   87. A method for preventing a thrombotic event in a patient with end stage renal disease, comprising parenterally administering to the patient a therapeutically effective amount of a product selected from the compounds defined any of paragraphs 1 to 75.
   88. A method for preventing venous thromboembolic events in a patient receiving chemotherapy through an indwelling catheter, comprising administering to the patient a therapeutically effective amount of a product selected from the compounds defined any of paragraphs 1 to 75.
   89. A method for preventing thromboembolic events in a patient undergoing a lower limb arterial reconstructive procedure, comprising parenterally administering to the patient a therapeutically effective amount of a product selected from the compounds defined any of paragraphs 1 to 75.
   90. A method of inhibiting platelet procoagulant activity, comprising parenterally administering to a mammal at risk of, or suffering from, arterial thrombosis a therapeutically effective amount of a product selected from the compounds defined any of paragraphs 1 to 75.
   91. A method of paragraph 90 wherein the disease is an acute coronary syndrome.
   92. A method of treating by way of therapy or prophylaxis an arterial disease selected from acute coronary syndromes, cerebrovascular thrombosis, peripheral arterial occlusion and arterial thrombosis resulting from atrial fibrillation, valvular heart disease, arterio-venous shunts, indwelling catheters or coronary stents, comprising parenterally administering to a mammal a therapeutically effective amount of a product selected from the compounds defined any of paragraphs 1 to 75.
   93. A method of paragraph 103 wherein the disease is an acute coronary syndrome.
   94. The use of a compounds defined any of paragraphs 1 to 75 for the manufacture of a parenteral medicament for a treatment recited in any of paragraphs 88 to 93.
   95. A pharmaceutical formulation comprising a combination of (i) a compound defined any of paragraphs 1 to 75 and (ii) a further pharmaceutically active agent.
   96. A pharmaceutical formulation comprising a combination of (i) a compound defined any of paragraphs 1 to 75 and (ii) another cardiovascular treatment agent.
   97. A formulation of paragraph 96 wherein the other cardiovascular treatment agent comprises a lipid-lowering drug, a fibrate, niacin, a statin, a CETP inhibitor, a bile acid sequestrant, an anti-oxidant, a IIb/IIIa antagonist, an aldosterone inhibitor, an A2 antagonist, an A3 agonist, a beta-blocker, acetylsalicylic acid, a loop diuretic, an ace inhibitor, an antithrombotic agent with a different mechanism of action, an antiplatelet agent, a thromboxane receptor and/or synthetase inhibitor, a fibrinogen receptor antagonist, a prostacyclin mimetic, a phosphodiesterase inhibitor, an ADP-receptor (P₂ T) antagonist, a thrombolytic, a cardioprotectant or a COX-2 inhibitor.
   98. The use of a compound defined any of paragraphs 1 to 75 for the manufacture of a parenteral medicament for treating, for example preventing, a cardiovascular disorder in co-administration with another cardiovascular treatment agent.
   99. A medicament comprising a boronic acid which is a selective thrombin inhibitor and has a neutral aminoboronic acid residue capable of binding to the thrombin S1 subsite linked through a peptide linkage to a hydrophobic moiety capable of binding to the thrombin S2 and S3 subsites, the hydrophobic moiety comprising a fluorinated ring in its S3-binding part, or a salt, prodrug or prodrug salt thereof.
   100. A medicament of paragraph 99 wherein the boronic acid has a Ki for thrombin of about 100 nM or less.
   101. A medicament of paragraph 99 wherein the boronic acid has a Ki for thrombin of about 20 nM or less.
   102. A medicament comprising a sodium or calcium salt of Cbz-(R)-4-F-Phe-(S)-Pro-(R)-Mpg-B(OH)₂.
   103. A pharmaceutical product comprising a sealed container containing in the form of a finely divided solid, ready for reconstitution to form a liquid formulation, a therapeutically effective amount of a boronic acid formula (II) or a salt, prodrug or prodrug salt thereof: 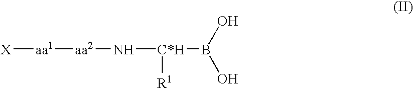
   where:
   X is H (to form NH₂) or an amino-protecting group;
   aa¹ is as defined in paragraph 9;
   aa² is an imino acid of S-configuration having from 4 to 6 ring members;
   C* is a chiral centre of R-configuration;
   and
   R¹ is a group of the formula —(CH₂)ₛ-Z, where s is 2, 3 or 4 and Z is —OH, —OMe, —OEt or halogen (F, Cl, Br or I).
   104. The product of paragraph 103 wherein:
   X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group;
   aa¹ is selected from (R)-4-F-Phe, (R)-4-F-Dpa, (R)-Cha and (R)-Dcha;
   aa² is Pro; and
   R¹ is 2-ethoxyethyl or 3-methoxypropyl.
   105. A pharmaceutical formulation adapted for oral or intravenous administration, whether directly or after combining with a liquid, and comprising
a) a first species selected from (a) boronic acids of formula (I) as defined in paragraph 1, (b) boronate anions thereof, and (c) any equilibrium form of the aforegoing (e.g. an anhydride), and combinations thereof; and
b) a second species selected from the group consisting of pharmaceutically acceptable cations having a valency n,
   wherein the formulation optionally has an observed stoichiometry of first to second species essentially consistent with a notional stoichiometry of 1:1 except where the second species is a metal ion having a valency of greater than 1, in which case the observed stoichiometry is essentially consistent with a notional stoichiometry of n:1.
   106. The formulation of paragraph 105 which has the characteristic that, after the formulation if not in an aqueous carrier is placed in one, it has a Ki for thrombin of about 20 nM or less.
   107. The formulation of any of paragraphs 77 to 79, 95 to 97, 105 or 106 or a medicament of any of paragraphs 99 to 102 which is in unit dosage form.
   108. The reaction product of an acid of any of paragraphs 1 to 27 and a base of any of paragraphs 40 to 53.
   109. The reaction product of an acid of any of paragraphs 1 to 27 and an amino sugar or a basic amino acid.

1. A compound selected from boronic acids of formula (I), and pharmaceutically acceptable salts, prodrugs and pharmaceutically acceptable prodrug salts thereof: 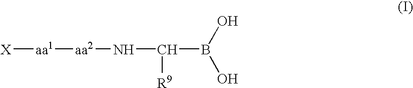
wherein
X is H (to form NH₂) or an amino-protecting group;
aa¹ is an amino acid residue having a side chain selected from formula (A) and (B):
—(CO)ₐ—(CH₂)_{b}-D_{c}-(CH₂)_{d}-E (A)
—(CO)ₐ—(CH₂)_{b}-D_{c}-Cₑ(E¹)(E²)(E³) (B)
wherein
a is 0 or 1;
e is 1;
b and d are independently 0 or an integer such that (b+d) is from 0 to 5 or, as the case may be, (b+e) is from 1 to 5;
c is 0 or 1;
D is O or S;
E is a saturated or unsaturated cyclic hydrocarbyl group; and
E¹, E² and E³ are each independently selected from the group consisting of 5-6 membered saturated or unsaturated hydrocarbyl rings, or one of E¹, E² and E³ is hydrogen and the other two are a said hydrocarbyl ring,
and wherein E, E¹, E² and E³ are halogenated;
aa² is a residue of an amino acid which binds to the thrombin S2 subsite; and
R⁹ is a straight chain alkyl group interrupted by one or more ether linkages and in which the total number of oxygen and carbon atoms is 3, 4, 5 or 6 or R⁹ is —(CH₂)ₘ-W where m is from 2, 3, 4 or 5 and W is —OH or halogen.

2. A compound of claim 1 wherein R⁹ is an alkoxyalkyl group.

3. A compound of claim 1 wherein E, E¹, E² and E³ are each independently selected from the group consisting of halogenated 6-membered rings.

4. A compound of claim 1 wherein a and c are both 0 and (a+b+c+d) and (a+b+c+e) are 1, 2 or 3.

5. A compound of claim 4 wherein aa¹ is of (R)-configuration, aa² is of (S)-configuration, and the fragment —NHCH(R⁹)—B(OH) is of (R)-configuration.

6. (canceled)

7. A compound of claim 1 wherein E, E¹, E² and E³ are fluorinated.

8. A compound selected from boronic acids of formula (II), and pharmaceutically acceptable salts, prodrugs and prodrug salts thereof: 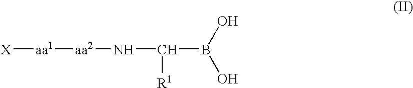
where:
X is H (to form NH₂) or an amino-protecting group;
aa¹ is an amino acid having a side chain which is C₁-C₅ alkyl substituted by one or two moieties selected from fluorophenyl, cyclohexyl and fluorocyclohexyl;
aa² is an imino acid having from 4 to 6 ring members;
R¹ is a group of the formula —(CH₂)ₛ-Z, where s is 2, 3 or 4 and Z is —OH, —OMe, —OEt or halogen.

9. A compound of claim 8 wherein aa¹ is selected from 4-F-Phe, 4-F-Dpa, 4-F-Dcha and 4-F-Cha.

10. A compound of claim 8 wherein aa² is a residue of an imino acid of formula (IV) 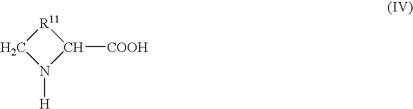
where R¹¹ is —CH₂—, —CH₂—CH₂—, —CH═CH—, —S—CH₂—, —S—C(CH₃)₂— or —CH₂—CH₂—CH₂—, which group, when the ring is 5- or 6-membered, is optionally substituted at one or more —CH₂— groups by from 1 to 3 C₁-C₃ alkyl groups, and optionally aa² is an (S)-proline residue.

11. A compound of claim 8 wherein aa¹ is of (R)-configuration and/or aa² is of (S)-configuration and/or the fragment —NH—CH(R¹)—B(OH)₂ is of (R)-configuration.

12. A compound of claim 8 wherein R¹ is 2-bromoethyl, 2-chloroethyl, 2-methoxyethyl, 3-bromopropyl, 3-chloropropyl or 3-methoxypropyl.

13. A compound of claim 8 where X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by one or more halogens, and/or by 1, 2 or 3 substituents selected from amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group.

14. A compound of claim 8 wherein the boronic acid is of formula (VIII):
X—(R)-4-F-Phe-(S)-Pro-Mpg-B(OH)₂ (VIII).

15. A compound of claim 1 which is in the form of a base addition salt of the boronic acid.

16. A compound of claim 15 which comprises a salt of the peptide boronic acid with an alkali metal or a strongly basic organic nitrogen-containing compound.

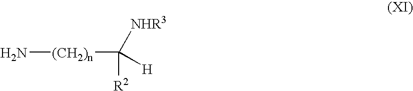

17. A compound of claim 15 which comprises a salt of the boronic acid with a metal.

18. A compound of claim 17 wherein the metal comprises an alkali metal salt.

19. A compound of claim 15 which comprises boronate ions derived from the peptide boronic acid and has a stoichiometry consistent with the boronate ions carrying a single negative charge.

20. A pharmaceutical formulation comprising a compound of claim 1.

21. A pharmaceutical formulation of claim 20 which is adapted for intravenous administration or for subcutaneous administration.

22. A pharmaceutical formulation of claim 20 which is adapted for oral administration.

23-24. (canceled)

25. A medicament comprising a salt, sugar ester or other soluble derivative of a boronic acid which is a selective thrombin inhibitor and has a neutral aminoboronic acid residue capable of binding to the thrombin S1 subsite linked to a hydrophobic moiety capable of binding to the thrombin S2 and S3 subsites, the hydrophobic moiety comprising a fluorinated ring in its S3-binding part and the salt comprising a cation having a valency n and having an observed stoichiometry consistent with a notional stoichiometry (boronic acid:cation) of n:1.

26. A method for making a product, comprising: contacting a boronic acid as defined in claim 1 with a pharmaceutically acceptable base to form the product.

27. The method of claim 26 which further comprises formulating the product into a pharmaceutical formulation.

28. A method of inhibiting thrombin in the treatment of a disease, comprising administering to a mammal an effective amount of a compound of claim 1.
